(19) United States
(12) Patent Application Publication
(10) Pub. No.: US 2004/0171089 A1
(43) Pub. Date: Sep. 2, 2004

(54) High-throughput screening assay for identifying substances capable of modulating cell survival and/or proliferation

(76) Inventors: Barbara Ann Spruce, Perthshire (GB); Jeremy John Lambert, Fife (GB); Peter Downes, Fife (GB); Nicholas Reid Leslie, Kinross (GB)

Correspondence Address: MYERS BIGEL SIBLEY & SAJOVEC, PO BOX 37428, RALEIGH, NC, 27627, US

(21) Appl. No.: 10/473,295

(22) PCT Filed: Apr. 2, 2002
(86) PCT No.: PCT/GB02/01523

(30) Foreign Application Priority Data
Mar. 31, 2001 (GB) 0108143.9

(51) Int. Cl. G01N 33/53; G01N 33/574

(52) U.S. Cl. 435/7.23

(57) ABSTRACT

The present invention relates to an assay particularly suited as a high-throughput screening assay for identifying novel compounds for use, for example, as anti-tumour, angiogenesis-modulating and/or anti-inflammatory agents, the assay being based on determining calcium levels within normal and abnormal cells.

### FIELD OF THE INVENTION

The present invention describes an assay particularly suited as a high-throughput screening assay for identifying novel compounds for use, for example, as anti-tumour, angiogenesis-modulating and/or anti-inflammatory agents.

### BACKGROUND

Agents which have a wide therapeutic index, that is to say, have clear selectivity of effect on diseased compared to normal cells have potential as drugs with low toxicity. Screening methods to identify novel drug candidates are often designed to identify interaction between the compound and its molecular target which can in some cases be a poor predictor of functional effect on living cells. The development of screening methods for compounds which have potential to modulate the cell's intrinsic death programme (apoptosis) is further complicated as this process is notoriously stochastic: timing of engagement of the apoptotic programme, even in a pure population of cultured cells, is extremely variable. Furthermore, decision-making events can often precede engagement of the programme by hours or even days. This makes it difficult to design a rapid, robust high throughput screen for novel agents which modulate the apoptotic programme.

Changes in intracellular calcium are recognised to be linked to the induction of apoptosis; however, the relationship between intracellular calcium flux and engagement of the apoptotic programme is complex. For example, it has been reported that the anti-apoptotic protein Bcl-2 can preserve concentrations of calcium within the lumen of the endoplasmic reticulum (He et al 1997 JCB Vol 138, pp1219-1228); conversely, another report describes a reduction in luminal ER calcium concentrations in response to Bcl-2 (Foyouzi-Youssefi et al 2000 PNAS Vol 97, pp5723-⁵⁷²⁸). The relationship between intracellular calcium and apoptosis is further complicated by reports that elevations in cytosolic calcium ions can be either pro- or anti-apoptotic (Bian et al. 1997 Am. J. Physiol. Vol. 272, C1241-1249; Nocotera and Orrenius 1998 Cell Calcium Vol. 23, pp173-180; Szalai et al 1999 EMBO J Vol. 18, pp6349-6361). Thus, on the basis of the prior art, the relationship between changes in intracellular calcium and apoptosis induction does not readily allow apoptotic outcome to be predicted by those skilled in the art on the basis of changes in intracellular calcium alone.

There is a known link between sigma ligands and changes in intracellular calcium. A rise in cytosolic calcium in neuroblastoma cells (a neuronal tumour cell line) in response to sigma ligands was shown by Vilner and Bowen (2000 JPET Vol 292 pp900-911), an effect that was mediated specifically through the sigma-2 receptor subtype. Hayashi and co-workers (JPET 2000 Vol 293 pp788-798) describe potentiation of bradykinin-induced calcium release with sigma-1 agonists, also in neuroblastoma cells; in contrast, the sigma-1 agonist (+) pentazocine blocked a potassium depolarisation-induced rise in cytosolic calcium. Thus, the relationship between sigma ligands and mobilisation of intracellular calcium is complex and unclear.

A link between sigma ligand-induced increase in intracellular calcium and apoptosis induction has also been proposed. A specific sigma ligand, reduced haloperidol (a metabolite of haloperidol which is selective for sigma receptors, unlike the parent compound) has been shown to induce a rise in cytosolic calcium in breast and colon carcinoma cell lines and it was proposed that this may be a “trigger” to apoptosis in these cells (Brent et al 1996 BBRC Vol 219 pp219-226). It is clear from the current art, however, that such a conclusion cannot be drawn as the relationship between mobilisation of calcium and apoptosis induction is more complex than was previously thought.

In WO 00/00599 (Spruce et al) it was disclosed that sigma ligands, including the sigma antagonistic ligand rimcazole, induce apoptosis and inhibit cell proliferation in a range of tumour cell lines. Sigma ligands act at least in part by provision of a pro-apoptotic switch to NF-kappaB which predicts inhibition of diseased cells associated with disordered inflammation where inappropriate activation of NFkappaB in its anti-apoptotic mode is known. Sigma ligands which are toxic to tumour cells have little or no effect on viability or proliferation of many normal cells; an exception is the lens epithelial cell type, a “self-reliant” cell. This contrasts with most inducers of apoptosis which are toxic to normal as well as diseased cells. The sparing of normal cells by sigma ligands predicts a wide therapeutic index—a measure of selectivity of effect on diseased compared to normal cells (further exemplified in WO 00/00599 by in vivo demonstration of tumour inhibition with no overt evidence of toxicity). The therapeutic potential of sigma ligands in the treatment of cancer and disorders of inflammation was therefore claimed.

In GB 0007842.8 (Spruce et al.) it was disclosed that an additional subset of (non-tumorous) cells is unduly susceptible to the inhibitory effects of sigma ligands: the microvascular endothelial cell, a cell type involved in response to injury and which also gives rise to new vessels associated with tumours; this so-called “angiogenic” response is crucial for tumour growth and survival. This selective inhibitory effect on microvascular endothelial cells was surprising as these cells are not self-reliant since they are sustained by provision of angiogenic factors produced by other cell types, for example fibroblasts and tumour cells; furthermore, they are not themselves tumorigenic. The ability of microvascular endothelial cells to disregard their microenviroment and survive in unfavourable conditions (due to possession of a greater degree of survival “autonomy” than most normal cells) was proposed as the basis for the selective effect on these cells. This was therefore a non-obvious extension of previous ideas. The direct but selective targeting of microvascular endothelial cells by sigma ligands, including sigma-1 antagonistic ligands, predicts application of sigma ligands to diseases associated with disordered angiogenesis including cancer (where adjuvant treatment may be specially indicated by the direct inhibition of tumour-associated vasculature in addition to the tumour cells themselves); also, non-cancer applications of angiogenic inhibition such as diabetic retinopathy and psoriasis, particularly in light of low predicted toxicity of the compounds. Conversely, sigma-1 agonists, or sigma antagonists to alternative receptor subtypes, are indicated for promotion of angiogenesis such as in promotion of wound or ulcer healing, or of a collateral vascular circulation as in ischaemic conditions.

In GB0007842.8 evidence is provided that the sigma-1 receptor in microvascular endothelial cells mediates an anti-apoptotic effect; and that abrogation of sigma-1-mediated survival leads to death of these cells. Yet Vilner and Bowen (1997 Soc of Neuroscience abstracts, Vol 23 p2319) describe a pro-apoptotic effect of sigma-2 agonists which indicates that the sigma-2 receptor behaves oppositely to the sigma-1 receptor in that it mediates a pro-apoptotic effect. A clear distinction is that Vilner and Bowen describe apoptosis induction by sigma-2 agonists in both tumour and primary cells (including cerebellar granule neurons, cells known to possess sigma binding sites). Sigma-2 agonists, at least on their own, would therefore be of less therapeutic value than sigma-1 antagonists which selectively kill tumour and endothelial cells but spare most other normal cells. Nevertheless, it has been predicted (WO 00/00599) that a combination of a sigma antagonist to one receptor subtype and a sigma or opioid agonist to a different receptor subtype, would provide an enhanced therapeutic effect. It should be said also, that the precise molecular identity of the sigma-2 receptor has not been confirmed. The gene encoding the sigma-1 receptor alone has been cloned (Kekuda et al. 1996 Biochem Biophys Res Comm Vol 229 pp553-558); the classification of remaining sigma receptor subtypes (of which there are proposed to be at least 3) must therefore remain provisional until their precise molecular identities have been confirmed.

Given this complex relationship between sigma ligands and apoptosis induction, standard methods of screening to identify sigma ligands which might be of therapeutic value in the context of cancer, disordered angiogenesis and/or inflammatory disease would be insufficient on their own. For example, in WO 00/00599 it was proposed that standard radioligand binding assays to detect affinity of a compound to sigma-1 and sigma-2 sites could be used to identify novel sigma ligands. However, it is clear from the current art that this method on its own would be ineffective to identify the subset of sigma ligands which induce apoptosis since sigma-1 and sigma-2 receptors may be anti- or pro-apoptotic. The standard radioligand binding assay detects binding affinity of a compound to a specific receptor subtype but does not give any measure of whether a receptor is being activated (agonistic effects) or inhibited (antagonistic effects). The radioligand binding assay would not exclude therapeutically-effective sigma ligands; but it would not discriminate between functionally active and inactive compounds. It therefore remains a problem to identify novel compounds which have pro-apoptotic effects through activity at sigma sites. The situation is further complicated by the disclosure by Vilner and Bowen that sigma-2 agonists are toxic to normal (primary) neural cells as well as to neural tumour cells. Thus, the therapeutic potential of a sigma ligand would not even be indicated by an ability to induce apoptosis in a cell line.

The current art therefore does not readily permit design of a screening method to identify molecules which modulate cell survival and/or proliferation in diseased or undesirable cells only.

It is amongst the objects of the present invention to obviate and/or mitigate this by providing such a screening method.

### SUMMARY OF THE INVENTION

Broadly speaking, the present invention is based on the discovery that a subset of sigma ligands which induce apoptosis and/or inhibit cell proliferation cause a rapid and sustained rise in intracellular free calcium ions. This rise in calcium appears to be a predictor of selective apoptosis induction and/or proliferation inhibition in diseased or undesirable cells in response to these agents since it does not occur, or is substantially less, when normal (primary) cell types are exposed to the same sigma ligands at the same concentrations; this holds even when the primary cells are known to possess sigma binding sites.

For the purposes of this invention, “normal” cells are defined as normal (primary) cells with typical properties of survival and/or proliferation regulation; “abnormal” cells are defined as cells that are diseased and have abnormal properties of survival and/or proliferation regulation; abnormal cells are also cells that are present in healthy tissues but have atypical properties of survival and/or proliferation regulation and can contribute to disease. Abnormal cells according to these definitions would include tumour cells, microvascular endothelial cells and persistent inflammatory cells.

Thus, in a first aspect, the present invention provides a method for identifying a substance capable of modulating cell survival and/or proliferation, which method comprises:

a) providing a test substance to a cell or cell population that displays abnormal properties of survival and/or proliferation regulation and to a cell or cell population that displays normal (as in typical) properties of cell survival and/or proliferation regulation;

b) observing an effect said test substance has on a level, either absolute or relative to a resting or other reference value, of cytosolic calcium within the abnormal and normal cells; and

c) comparing any effects said test substance has on the level, or relative level (as defined in (b), of cytosolic calcium within the abnormal (diseased or atypical) and normal (typical) cells, so as to identify agents which affect the level or relative level of cytosolic calcium within the abnormal cells whilst minimally or substantially not affecting the level or relative level of cytosolic calcium within the normal cells.

Without wishing to be bound by theory the present inventors have observed an acute elevation in cytosolic calcium in cultured tumour cells exposed to sigma (antagonistic) ligands; the magnitude and duration of calcium elevation correlates with apoptotic outcome. Induction of a calcium spike with an antagonist could be due to acute derepression of a pro-death mediator which is constitutively in place. In stark contrast to tumour cells, primary cell types, at least some of which are known to possess sigma binding sites show little or substantially no increase in cytosolic calcium when exposed to sigma (antagonistic) ligands. The present inventors therefore provide for the first time an assay which allows the identification of agents which selectively modulate cell survival and/or proliferation in diseased cells, or undesirable cells which contribute to disease, only. Thus, agents which, for example, result in a significant increase in cytosolic calcium in normal (typical) as well as diseased an/or atypical cells would be predicted to be of little or no therapeutic value. However, agents which result in a significant increase in cytosolic calcium, only in cells which display abnormal survival and/or proliferation regulation (ie. do not result in a substantial alteration in cytosolic calcium in cells with normal—typical—survival and/or proliferation regulation) would be predicted to be of good therapeutic value.

Providing candidate substances to cells may be performed by for example addition to the extracellular saline solution as defined for example in the Materials and Methods or by employing methods of enforced cellular internalistion such as liposomes or electroporation. The assay is typically carried out in vitro on live cells. The cells are typically cells of 1) a mammalian continuous cell line; 2) very low passage (see GB 0007842.8 for explanation) primary cell cultures such as adult dermal microvascular endothelial cells (atypical survival properties) or adult dermal fibroblasts (typical survival properties) or rodent cerebellar granule neurones (typical survival properties). Candidate substances may be added at suitable concentrations and/or ranges, for example between 1 nM and 0.1 mM. The present inventors observe that addition of certain substances, such as known sigma-1 antagonists results in an increase in cytosolic calcium levels which occurs rapidly, within seconds to minutes. This is important when carrying out a high-throughput screen as the quicker a response can be detected, the more substances can be screened. Without wishing to be bound by theory, it is thought that a sustained increase in calcium levels is a particular property of sigma-1 antagonists in at least some cell types which may contribute importantly to the induction of apoptosis. In this matter the assays of the present invention may be run for an extended period of time, for example, 10-60 minutes, say 15-30 minutes, to observe if any modulation in calcium level is maintained over the time course of the assay.

“Modulating cell survival and/or proliferation” is understood to mean any alteration in the cell survival and/or proliferation characteristic as displayed by a particular cell type. As such this could mean for example induction of programmed cell death (ie. apoptosis) and/or induction or inhibition of cell proliferation. Thus, for example, in tumour cells, which display abnormal cell proliferation, “modulating cell-survival and/or proliferation” may result in induction of apoptosis and/or inhibition of proliferation.

In another aspect of the invention, modulating cell survival and/or proliferation is taken to mean a promotion of cell survival and/or proliferation, such as to promote beneficial angiogenesis as would be of use for example in wound or ulcer healing or to promote collateral circulations in vascular ischaemia or after infarction. In this aspect, the assay will be applied to the identification of agents which inhibit the calcium rise in respect to, for example, known sigma-1 antagonists, as are described in this invention. Agents with this property could be deduced to restore the repression to the pro-death mediator kept “in check” by the sigma-1 receptor (GB 0007842.8). In this way, survival and proliferation of microvascular endothelial cells in adverse (survival factor-poor and/or hypoxic, for example) circumstances would encouraged and beneficial new vessel networks could be established.

For the purposes of the present invention, cells which display abnormal properties of survival and/or proliferation regulation include tumour cells, of any origin and/or at any stage of malignancy; also, unusual subsets of “normal” cell types including lens epithelial and microvascular endothelial cells and other cells with unusual survival properties such as cartilaginous cells; and lastly cells in which NFKappaB is dysregulated in such a way as to model diseases of persistent inflammation such as arthritis, major organ inflammation such as chronic nephritis, and atheroscelerosis and Alzheimer's disease, or persistant inflammatory cells themselves. NFkappaB-dysregulated cells mimic tumour cells and microvascular endothelial cells in exhibiting undue reliance on sigma-mediated survival and are therefore classed as cells with atypical survival and/or proliferation regulation (WO 00/00599). Thus generally speaking, the assays of the present invention may be used for identifying anti-tumour, angiogenisis-modulating and/or anti-inflammatory agents.

Examples of normal (primary) mammalian cells with a finite life span and with typical properties of survival and proliferation regulation include human adult fibroblasts of dermal or other origin; mammary epithelial cells; prostate epithelial cells; cerebellar granule neurones (likely to be rodent in origin). For the purposes of this invention it is important that all such cells are examined at low passage (that is, within the maximum number of population doublings recommended by the supplier—such as Clonetics Inc—for that particular primary cell type—GB 0007842.8; or in the case of rodent culture, from freshly isolated tissue).

The level of cytosolic calcium within the normal and abnormal cells may be detected by methods known to the skilled addressee that monitor cytosolic calcium levels. Indicator dyes may be used, for example fluorescent probes (such as fura-2, indo-1, quin-2) show a spectral response upon binding calcium and it is then possible to detect changes in intracellular free calcium concentrations using for example fluorescence microscopy, flow cytometry and fluorescence spectroscopy. Most of the above fluorescent indicators are variations of the nonfluorescent calcium chelators EGTA and BAPTA (Cobbold and Rink,1987). Other examples are obtainable from Moleclar Probes, Oregon, USA.

Additionally, the present assays are particularly suited to the development of high-throughput screens where detection may be carried out using for example a CCD camera, a luminometer, or any other suitable light detection system. In this manner, cells may be provided for example in multi-well plates to which test substances and reagents necessry for the dtection of intracellular calcium may be added. Moreover, commercially available instruments such as “FLIPR-flumetric imaging based plate reader” (Molecular Devices Corp, Sunnyvale, Calif., USA) and “VIPR” voltage ion probe reader (Aurora, Bioscience Corp. CA, USA) may be used. New fluorescent indicators for calcium called “cameleons” may also be used and are genetically encoded without cofactors and are targetable to specific intracellular locations. These so-called “cameleons” consist of tandem fusions of a blue-or cyan-emitting mutant of the green fluorescent protein (GFP), calmodulin, the calmodulin-binding peptide M13, and an enhanced green- or yellow-emitting GFP. Binding of calcium makes calmodulin wrap around to M13 domain, increasing (Miyawaki et al 1997) or decreasing (Romoser et al 1997) the fluorescence resonance energy transfer between flanking GFPs.

Additionally, potentiometric optical probes may be used. Potentiometric optical probes measure membrane potential in organelles and in cells. In conjunction with imaging techniques, these probes can be employed to map variations in membrane potential along neurons and among cell populations with high spatial resolution and sampling frequency (Rohr & Salzberg 1994).

A substance which modulates cell survival and/or proliferation in an abnormal cell may do so in several ways. Without limiting the scope of the invention, candidate agents may act through action on sigma receptors, eg. sigma-1 receptor. As such the present assay may therefore be of use in discovering novel sigma receptor (eg. sigma-1) ligands which may be agonists or antagonists.

Suitable candidate substances may come from combinatorial libraries, peptide and peptide mimetics, defined chemical entities, oligonucleotides, and natural product libraries which may be screened for activity. In one possible approach the candidate substances may for example be used in an initial screen in batches of, for example 10 substances per reaction, and the substances of those batches which show a response modulation tested individually. Candidate substances which show activity in the assays of the present invention can then be further tested in other systems, such as in ligand binding assays known in the art and/or apoptosis assays, before testing in animals, such as mice, rats, rabbits, humans and the like for therapeutic efficacy. For example a further screen, or initial screen may employ standard radioligand binding assays in which a candidate substance is allowed to compete with radiolabelled specific sigma ligands for sites on isolated cellular membranes rich in sigma sites.

The ability of a candidate substance to induce apoptosis or be cytotoxic can be determined by administering a candidate compound to cells and determining if apoptosis is induced in said cells. The induction of apoptosis/cytotoxicity can be determined by various means eg. FACScan, MTS assay and assays of caspase activation. There are several techniques known to a skilled person for determining if cell death is due to apoptosis. Apoptotic cell death is characterised by morphological changes which can be observed by microscopy, for example cytoplasmic blebbing, cell shrinkage, intermucleosomal fragmentation and chromatin condensation. DNA cleavage typical of the apoptotic process can be demonstrated using TUNEL and DNA ladder assays.

Alternatively, it may be desired to prevent apoptotic cell death by administering a substance identifiable by the method of the invention. Several techniques known in the art for inducing apoptosis in cells may be used. For example, apoptosis may be induced by stress including UV exposure, growth factor deprivation and heat shock. The ability of the candidate substance to inhibit such apoptosis may be determined by comparing cells exposed to stress in the presence of the candidate substance with those exposed to stress in the absence of the candidate substance.

Thus, an additional aspect of the invention would be to apply the assay in a reciprocal manner—to identify sigma ligands (such as sigma-1 agonists) which are acting oppositely to those of use in the treatment of cancer; in this application, the agents would be used in diseases where excess apoptosis is a causative factor. These diseases would include neurodegenerative disorders (such as Parkinsons disease) and AIDS. As described above, in the context of angiogenesis-promotion, the assay would be applied as follows: test compounds would be coapplied with agents known to induce a calcium rise, such as known sigma-1 antagonists as defined in this invention. Test compounds which inhibit the sigma antagonist-induced calcium rise would be preducted to restore activity to the sigma-1 receptor and thus to promote its anti-apoptotic function (GB 0007842.8). Since the sigma-1 receptor behaves as a geneal repressor of cell death (GB 0007842.8) compounds identified in this way would be of general use to prevent or repress engagement of the apoptotic programme.

Compounds identified in this way could be of use to save remaining viable neurones after a cerebrovascular accident (“stroke”) or heart cells after a myocardial infarction or after diagnosis of chronic neurdegeneration (Alzheimers, Parkinsons etc) when neurones which have not yet committed to the death programme (irreversible once engaged) could be saved. Since the sigma-1 receptor may also be acting close to or coincident with the final common pathway to death it is even possible that agents which promote or mimic sigma-1 receptor function could arrest the death programme once it is engaged.

An potential application of the assays of the present invention may be to indicate whether sigma ligands can be used to enhance the sparing of normal tissues when conventional cytotoxics or radiation are administered. The present inventors have data that sigma and opioid ligands synergise with standard chemotherapeutics when administered to tumour cells. This may allow a lower dose of radiation or cytotoxics to be administered to patients when combined with sigma ligands and therefore achieve a greater sparing of normal tissue than with conventional regimes. However, it is possible that sigma ligands combine with conventional agents to attack normal tissue even though they are harmless on their own. The present screens (which compares effects on normal cells with for example tumour cells) will give a prediction of therapeutic index when conventional treatments are combined with sigma ligands. Desirable combinations of agents which can be progressed to preclinical and clinical trial may therefore be identified.

Other cells which may be screened include cells which for example have been modified to overexpress the cloned sigma-1 receptor cDNA, to see whether or not this will attenuate a modulation in the level of cytosolic calcium. It may be anticipated that the overexpressed protein would prevent derepression of a pro-death substance with which the sigma-1 receptor is physically or functionally coupled.

Moreover, antisense constructs designed against the gene sequence of the sigma-1 receptor may be added to cells in order to prevent expression of the sigma-1 receptor. This may be done to ascertain whether or not the sigma-1 receptor is required for a modulation in calcium levels. However, cells may die as sigma-mediated survival may be required to suppress death.

Moreover, the so-called RNAi technique (Zamore et al., 2000 Cell Vol 101 pp 25-33) may be used to prevent expression of the sigma receptor. RNAi has the potential to be more effective than the antisense approach.

Moreover, sigma receptor knockout cells eg. embryonic stem cells knocked out for endogenous sigma-1 receptor, in which wild-type and mutant forms of the sigma-1 receptor could then be overexpressed with and without transforming encogenes (and/or microvascular endothelial cell-specific genes) to recreate “sensitive cells” which display a calcium rise in response to sigma ligands.

In this way, a number of analyses may be used to characterise the molecular target and to confirm therapeutic potential of agents which were identified in the primary high throughput screen (anticipated to select out many compounds which are therapeutic “non-starters”).

One embodiment of the present invention is that novel ligands which specifically have an antagonistic effect at the sigma-1 receptor subtype may be identified through the assays of the present invention; these may have the therapeutic properties described above and may have enhanced activity. In GB0007842.8 it was disclosed that the endogenous sigma-1 receptor in microvascular endothelial cells mediates an anti-apoptotic effect; furthermore, that abrogation of this survival pathway caused the death programme to be selectively unleashed in these cells. This was exemplified by the ability of a panel of agents which are highly specific for the sigma receptor class and are generally deemed to be antagonistic, to induce death in these cells. Death/proliferation inhibition was completely prevented by administration of equimolar concentrations of a sigma-1 ligand, (+)-pentazocine, which is widely regarded as a prototype for the sigma-1 agonist class of compound; death induction by rimcazole and IPAG could therefore be deduced to be a consequence of antagonism at the sigma-1 receptor subtype, in microvascular endothelial cells.

Since filing of this invention 12 months ago, the art is now indicating a strong link between calcium-conducting channels and cancer. In particular, members of the transient receptor potential (TRP) family of non-selective cation channel proteins have been linked to both the promotion and suppression of tumours. For example, melastatin, a TRP-related protein, has previously been linked to suppression of melanomas. Downregulation of melastatin is correlated with metastatic potential of melanomas (Duncan et al. 1988; Cancer Res. Vol. 58 pp1515-1520). Moreover, successful treatment of melanoma is associated with upregulation of melastatin (Deeds et al. 2000 Hum. Pathol. Vol 31 pp 1346-1356). The ability of melastatin to elicit calcium influx was recently shown, confirming its function as a calcium-permeable channel (Shawn Xu et al. September 2001 PNAS Vol. 98 pp 10692-10697). These findings would lend support to our model that the sigma-1 receptor restrains a pro-death calcium flux mechanism whose derepression by sigma antagonists will have anti-tumour effects. Furthermore, we have shown that intracellular calcium responses induced by so-called sigma-1 antagonists are sensitive to extracellular calcium, from which a mechanism involved in calcium influx is inferred.

On the other hand, another TRP-related protein, TRP-P8 is suggested to be a tumour-promoter. Its expression is upregulated in many tumours including melanoma and breast, lung and colorectal carcinomas; whereas its expression in normal tissues is mostly low or absent (Tsavaler et al. May 2001 Cancer Research Vol. 61 pp 3760-3769). These workers suggest a role for TRP-P8 as a calcium channel protein which protects cancer cells from apoptosis and thus distinguishes it from melastatin. They further suggest that calcium channel blockers may be of use in the treatment of cancer.

Together, these data indicate that TRP proteins have a clear link to cancer but this is not straightforward as according to the art they may be both tumour-promoting and tumour-suppressing.

On the other hand, it is well recognised in the field of oncology that expression levels are not sufficient to predict the function of a given protein as either tumour suppressor (deduced from low levels in neoplasia) versus an oncogene (high levels in neoplasia). It is conceivable that members of the TRP family of channel proteins involved in calcium influx may be pro-apoptotic and tumour suppressive even when they are overexpressed in tumours.

We hypothesise that a mechanism that involves calcium influx is corecruited as an obligate pro-apoptotic safeguard in cells which possess a degree of self-reliance such as primary microvascular endothelial cells and lens epithelial cells; tumour cells are therefore similarly burdened. m this may be the price that tumour cells pay for acquiring a degree of self-reliance. Thus, high level expression of a calcium channel or other calcium flux mechanism in tumours may reflect a greater degree of self-reliant behaviour.

In their physiological role, some TRP proteins have been shown to be involved in calcium influx in response to depletion of intracellular calcium stores (Clapham et al June 2001 Nature Reviews in Neuroscience Vol 2 pp 387-396). We have determined that sigma antagonists elicit calcium influx in tumour cells and microvascular endothelial cells. But it is notable from our data that the calcium rise in tumour cells induced by sigma antagonists is immediately sensitive to withdrawal of extracellular calcium. It is therefore possible that in tumour cells, a TRP channel may be decoupled from intracellular stores such that it can be activated (or derepressed) even when intracellular calcium levels are high. Microvascular endothelial cells display a slightly different behaviour in revealing an initial rise in intracellular calcium when extracellular calcium is nominally absent (see below); however, the rise in calcium is not sustained. Thus, microvascular endothelial cells also display calcium influx in response to sigma antagonists but in these cells, sigma antagonists may have an additional role in causing initial release of calcium from intracellular stores. (This may indicate a lesser degree of deregulation since microvascular cells are not themselves tumour cells). Therefore, to identify agents that will unleash suicide in tumour cells and other undesirable cells, a particular embodiment of the calcium imaging method is to perform the assay in the presence and nominal absence of extracellular calcium in order that calcium influxcan be confirmed. Removal of extracellular calcium will either abolish or prematurely terminate the rise in intracellular calcium evoked by sigma antagonists.

It has also become apparent in the intervening period since this invention was filed that agents which do not bind directly to the sigma receptor can cause an increase in intracellular calcium and that this increase in intracellular calcium is necessary for death. This includes molecules known to have anti-tumour properties such as tumour necrosis factor (TNF) alpha. TNF has been shown to induce calcium dependent death in tumour cells. Furthermore, the TNF-induced calcium rise and death were abolished by antisense oligonucleotides to a member of the TRP family, thereby implicating a TRP molecule in TNF-induced death (Hara et al 2002 Molecular Cell Vol. 9 pp163-173). The importance of this finding lies in the fact that TNF is known to be upregulated in tumour cells as a result of for example activation of NF-kappaB in response to treatment with ionising radiation and some chemotherapeutics. Thus, many anti-cancer agents could be anticipated to have the potential to cause release of TNF and thereby TRP-mediated capacitative calcium entry. But it is also known that many of these agents are toxic to normal cells. This includes TNF whose early promise as an anti-tumour agent was dispelled as a result of unacceptable toxicity to normal tissues. Thus, the involvement of TRP-mediated calcium influxmay be in itself insufficient to identify agents that will selectively kill tumour cells but spare or have greatly reduced effects on most normal cells. One possible explanation is that the molecular identity of the TRP (or other) calcium channel may be important in determining cell-selective calcium entry and death. But until such time as this is determined, the demonstration of calcium influxon its own appears insufficient to predict maximally selective anti-tumour action.

On the other hand, the involvement of at least one TRP channel in TNF (and therefore other anti-cancer agent)—induced death lends strong support to our prediction that calcium imaging will be of use to identify combinations of sigma ligands with known classes of chemotherapeutics and ionising radiation regimes that will be most potent in their anti-tumour action.

The potential insufficiency of calcium imaging on its own to identify agents that have the best potential to discriminate between tumour or undesirable cells on the one hand and normal or desirable cells on the other hand, suggests a need for a way in which the assay of the invention can be modified in order to take this into account.

One potential solution is to perform the assay using a 3-way comparison of tumour cells and one type of atypical cell such as microvascular endothelial cells against normal (typical) cells. Agents that substantially raise cytosolic calcium in both tumour cells and at least one other type of primary atypical cell (with self-reliant behaviour) such as microvascular endothelial cells or lens epithelial cells, but do not substantially raise calcium in normal (as defined herein) cells will be deemed to have a high probability to be non-toxic.

It is also conceivable that non-toxic anti-tumour compounds may be identified by a comparison of (wholly or partially) self-reliant non-tumour cells against normal cells (a 2-way comparison) alone. This is because activity against self-reliant cells can conceivably be expected to predict anti-tumour activity associated with a low degree of toxicity.

One potential solution is to perform sigma radioligand binding assays either as a preceding or subsequent step to calcium imaging in order that the method would be confined to the identification of agents that bind either directly to the sigma receptor, or to a binding site that is allosterically coupled to a sigma binding site. Interpretation is however complicated by the possibility that multiple binding pockets may exist even within the sigma receptor itself. Therefore, until the precise binding pocket(s) on the sigma receptor macromolecule that mediate its involvement in cell survival (and in particular in autocrine survival) have been identified, such assays have limitations (for example in extrapolation from apparent binding affinity in conventional ligand binding assays).

Faced with these complexities, one potential solution was to turn to the identification of signalling molecules associated with the calcium rise, in the hope of identifying a biochemical readout from the calcium rise which might help to discriminate between the mechanisms of calcium influx. It may therefore be possible to identify those agents that will be most effective against diseased and undesirable cells whilst being maximally sparing of normal cells, without needing at this stage to know precisely the mechanism whereby cytosolic calcium is raised.

There are much data to indicate to a skilled man that changes in intracellular calcium are linked to changes in phosphatidylinositol signal transduction pathways (for example, Clapham et al 2001 Nature Reviews in Neurscience Vol 2 pp 387-396 and references therein; Berridge et al 1998 Nature Vol 395 pp645-648 and references therein; Kim et al 1999 J Biol Chem Vol 274 pp26127-26134). This includes known links between PKB/Akt and calcium (Worrall and Olefsky 2002 Molecular Endocrinology, Vol. 16 pp 378-389; Crossthwaite et al 2002 J. Neurochem Vol 1 pp24-35; Tang et al 2002 J Biol Chem Vol 277 pp 338-344).

Thus, in the light of the calcium work the present invemtors considered conducting assays that would determine changes in the activity of enzymes phospholipase C (PLC) and the protein kinase B (PKB/Akt) enzyme. (The PKB enzyme has two accepted names in the art: PKB and Akt). Furthermore, as discussed elsewhere in this invention, such pathways have known links to cell survival regulation and were therefore likely participants in calcium-regulated cell viability.

Assays to detect activity of phospholipase C and activity of the PI3 kinase/PKB pathway can be conducted in living cells using a technology based on so-called pleckstrin homology PH domain fusion proteins between specific PH domains and a fluorescent or other locatable tag such as green fluorescent protein (GFP). In the case of PLC activation (or events suggestive of PLC activation) this can be detected using a so-called GFP-PHPLC delta protein (as described elsewhere). In the case of PI3K/PKB activity, a so-called GRP-1 protein is employed (as described elsewhere). Platform technologies (Taylor et al., 2000, Current Opinion in Biotechnology, 12, pp75-81 and Kapur, et al., 1999, Biomedical Microdevices, 2, pp99-109)) are now available to exploit this technology which render it amenable currently to at least medium throughput screening.

Assays that employ so-called PH domain protein technology are of particular value in the likely anticipation that some agents of the invention are more susceptible to levels of extracellular survival factors (as has been described previously in WO 9606863, WO 000599 and WO 0174359). This is because assays that employ the so-called GFP-PH domain fusion protein technology (as described in detail elsewhere in this invention) can be conducted either in the presence or absence of high serum (unlike calcium imaging which is restricted to execution in defined saline solution). It is conceivable that agents which have greater activity in low serum compared to high serum conditions will be of more value in the clinic. This is particularly relevant for potential anti-tumour agents since many cancers in vivo are starved of nutrients. And also for anti-angiogenic agents since, again, the angiogenic response occurs under conditions of nutrient deprivation.

The GFP-PH domain assays are also of particular value when there is an advantage in conducting the assays over a longer time course than is usual for calcium imaging. This could be of value when testing cells which are slower to die than others and whose point of commitment to death may therefore be later. GFP-PH domain assays can be conducted for periods up to 48-72 hours approximately. There are many examples in the literature (and also in WO 9606863, WO 000599 and WO 0174359) of wide differences in the timing of engagement of the death programme. These assays would be of particular value in the context for example of low to medium throughput screening and where information obtained over a longer time course could be an advantage, for example in being less cell type-sensitive.

The application of GFP-PH domain assays (to detect PLC activation) to identify non-toxic anti-tumour agents is not obvious from prior art since most agents that activate PLC are not of therapeutic value. However, this invention provides information that enables the person skilled in the art to conduct the PLC activation assays in such a way that agents of the invention (namely agents that display selective toxicity for cells that possess a degree of self-reliance are identified; these agents include but are not restricted to sigma antagonists) may be identified from the assays either alone or as an adjunct to other assays of the invention (such as calcium imaging and PKB/Akt assays). In particular, the assay is conducted with different concentrations of agents over a time course which can if necessary be extended up to 48-72 hours. It may also be conducted in the presence and nominal absence of calcium (cells cultured in nominally calcium free medium or medium to which calcium chelators have been added). It can also be performed in low and high serum conditions, for example.

On the other hand, it is not a novel idea to identify potential anti-tumour agents using methods that detect inhibition of PKB/Akt activity. Thus, PH domain protein technology to detect activity of the pathway in which this enzyme participates as an assay on its own is not a method of this invention. What is however non-obvious even to the skilled man is to conduct assays of PKB activity in combination (simultaneously or otherwise) with PLC activation assays and one or more other assays of the invention. for example calcium imaging and sigma radioligand binding assays.

Thus, in a further aspect, the present invention provides a method for identifying a substance capable of modulating cell survival and/or proliferation, which method comprises:

c) providing a test substance to a cell or cell population that displays abnormal properties of survival and/or proliferation regulation and to a cell or cell population that displays normal (as in typical) properties of cell survival and/or proliferation regulation;

d) observing an effect said test substance has on a level, either absolute or relative to a resting or other reference value, of PLC activation within the abnormal and normal cells; and

c) comparing any effects said test substance has on the level, or relative level (as defined in (b), of PLC activation within the abnormal (diseased or atypical) and normal (typical) cells, so as to identify agents which affect the level or relative level of PLC activation within the abnormal cells whilst minimally or substantially not affecting the level or relative level of PLC activation within the normal cells.

In this invention it is shown that the rise in intracellular calcium in response to IPAG occurs within seconds. The inventors considered the possibility that the rise in cytosolic calcium is secondary to a phospholipase C (PLC)-mediated increase in IP3 levels, which can occur in a subsecond time frame. IP3, through binding to the IP3 receptor, would then lead to release of calcium from intracellular stores. However, this appeared unlikely as it was observed that the increase in intracellular calcium in tumour cells is abolished if extracellular calcium is withdrawn. This therefore suggested that, at least in tumour cells, the cytosolic calcium rise is initiated by calcium influx and that intracellular calcium stores are perhaps less important as the source of the initial calcium rise. It has nonetheless been suggested that in at least some cells, intracellular calcium stores can be very closely coupled to capacitative calcium entry channels. Therefore, it might be difficult to temporally resolve depletion of intracellular stores if this is closely followed by capacitative calcium entry. Indeed, the present inventors have shown that in microvascular endothelial cells exposed to sigma antagonists, the initial rise in cytosolic calcium is due to release of calcium from intracellular stores (since it is not sensitive to withdrawal of extracellular calcium); the subsequent maintenance of the elevated calcium is however dependent on calcium influx since this is sensitive to nominal withdrawal of extracellular calcium). (“nominal” calcium withdrawal in this context means exposure of cells to a saline solution not containing added calcium but to which calcium chelators have not been added. Thus, small amounts of calcium are likely to be present from glassware, for example.)

The inventors also considered the possibility that calcium influx might cause, rather than be the consequence of, PLC activation. According to this model, the rise in calcium would be required for activation of PLC and subsequent generation of IP3 which could then feed forward to amplify the calcium signal through secondary release from intracellular stores. Although the activation of all isoforms of PLC is calcium dependent to some extent (Clapham et al. 2001, as cited above), the delta isoform of PLC is particularly calcium dependent and can be activated by calcium influx(Kim et al. 1999 J. Biol. Chem. Vol. 274 pp 26127-26134). The present inventors went on to show that so-called sigma antagonists, agents that selectively unleash cell suicide in tumour cells and other cells that possess a degree of self-reliant behaviour, cause relocalisation of a GFP-PHPLC delta (as described elsewhere in this invention) fusion protein and also elevate IP3 and IP4 levels, effects that strongly implicate the activation of PLC. Hereafter, the activation of PLC is taken to mean these biochemical changes. Sigma antagonists cause the activation of PLC and this activation appears to require extracellular calcium. In contrast, sigma-1 agonists and sigma-2 agonists do not activate PLC.

Whichever type of calcium flux mechanism is involved, the outcome in all cases is activation of PLC in an extracellular calcium-regulated manner—most likely therefore to be the delta isoform of PLC—which therefore represents a biochemical readout of the calcium response to sigma antagonists. What is also a potential defining signature, is that the ligand concentration affects the timing of onset but not the magnitude of the outcome in response to PLC activation

The inventors exemplified the activation of PLC using 2 methods: Firstly, time lapse fluorescence imaging of cells into which a cDNA, encoding a hybrid protein consisting of the pleckstrin homology (PH) domain of PLC delta fused to green fluorescent protein (GFP), had been introduced. Secondly, quantitative HPLC assay of specific inositol phosphates and inositol phospholipids. The former method is amenable to scale up for medium and potentially high-throughput screening and is thus a useful adjunct to high throughput calcium imaging as a means to discriminate between different modes of calcium influx that might determine cell-selective suicide.

Further embodiments of the invention would be to perform one or both of calcium imaging and assays for PLC activation, optionally together with sigma radioligand binding assays (in no particular temporal order). Agents would be selected if: at concentrations in the range anywhere from micromolar to subnanomolar, they displace radiolabelled prototypic sigma ligands from cell membranes known to possess sigma receptors; and in addition, evoke one or both of the following: an extracellular calcium-sensitive rise in cytosolic calcium and extracellular calcium-dependent activation of PLC. Sigma radioligand binding assays as previously described in this and preceding inventions (WO 000599 and WO 0174359) will identify i) sigma ligands that interact directly with one or more binding pockets of sigma receptor subtypes; ii) agents that allosterically modulate the interaction of prototypic sigma ligands with sigma receptor subtypes. These agents are defined as sigma ligands for the purposes of the invention. Embodiments that incorporate sigma radioligand binding assays may be advantageous in increasing the likelihood that non-toxic, therapeutically effective compounds are identified. But embodiments that lack sigma radioligand binding assays still have the potential to identify agents that directly or indirectly activate a cytosolic calcium-raising mechanism and/or PLC in such a way that cell-selective suicide, as engaged by sigma antagonists, is unleashed.

An additional discovery made by the present inventors is that sigma antagonists also have the capacity to inhibit protein kinase B (PKB/Akt), an enzyme that is known to play an important role in cell survival and is frequently deregulated in human cancer. In MDA MB 468 cells, absence of the PTEN tumour suppressor protein leads to elevated activity of PKB. Thus, sigma antagonists appear to have an ability to concomitantly unleash a calcium/PLC-dependent signalling pathway that may be pro-apoptotic; and furthermore, to inhibit PKB, a recognised pro-survival enzyme. The inventors exemplified the reduction in PKB activity using prototypic assays. The time course of PKB inhibition appears to correlate with activation of PLC, and is also dose-dependent.

It is conceivable that the calcium-dependent activation of PLC is an indirect cause of the inhibition of PKB, for example through reduction in PIP2 levels. Inhibition of PKB is therefore an additional potential readout of calcium effects induced by sigma antagonists.

The application of appropriate technologies that exploit this finding will have a use as an adjunct to calcium imaging. This is particularly so, given that analogous techniques to those described for PLC activation can be used to confirm inhibition of PKB activity in living cells and are therefore also amenable to scale up to medium and high throughput screening technologies. Specifically, a PH domain construct fused to GFP which binds PIP3 is introduced into living cells (by transfection of the cDNA, microinjection of the cDNA or protein or other internalisation methods such as liposomes). The PH domain protein (dubbed GRP-1: Gray, et al., 1999, Biochem. J., 344, pp929-936) binds specifically to PIP3 and is normally cytosolic. However, when PIP3 levels are elevated, (as in MDA MB 468 cells) the construct is localised to the membrane. When the PI3 kinase pathway is inhibited (as it would be after exposure to sigma antagonists) the Grp-1 protein is translocated to the cytosol. By the use of variants of GFP that emit fluorescent signals of different wavelengths, PH domain constructs could be used simultaneously to determine the concomitant activation of PLC and inhibition of PKB in living cells. MDA MB 468 cells are a particularly suitable model cell system to use for this embodiment of the invention as they lack PTEN and thus have elevated PKB activity

Elevation in the activity of PKB, due to absence of PTEN or for other reasons, is not however a universal feature of tumour cells. But the ability of sigma antagonists to inhibit PKB even when elevated above normal levels would predict that these agents would also be of value to inhibit PKB in tumour cells that retain regulation of PKB. Thus, the inhibition of this enzyme is likely to have broad relevance to cancer. But for the purposes of a screening assay to identify novel agents that also have this activity, a PTEN null cell line such as MDA MB 468 cells that possesses constitutively active PKB will be of particular use in the assay.

It is currently unknown whether all agents of the invention will concomitantly activate PLC and inhibit PKB. However, the inventors believe that calcium—dependent activation of PLC represents a hallmark which is common to agents of the invention. An assay to detect concomitant (not in the sense of simultaneous) inhibition of PKB is viewed at this stage as a non-obligate adjunct to calcium imaging and the assay for PLC activation (performed in the presence and absence of extracellular calcium). This represents a further embodiment of the invention.

Inhibitors of P13 kinase (and therefore PKB activity)—exist that do not activate PLC and that are not sigma ligands; these would include the compounds Wortmannin (Sigma) and LY-294002 (Calbiochem). To distinguish the agents of the invention from such agents and other PI3 kinase inhibitors, PI3 kinase pathway assays must be performed together with (in no particular temporal order) one or more of the following assays: calcium imaging, PLC activation and sigma radioligand binding assays, as have been described in this and preceding inventions (WO 000599 and WO 0174359). The invention is thereby confined to agents that score in at least one assay additional to the assay of PI3 kinase pathway activity. The present inventors also believe that by use of such a multi-layered approach to the screening technology, the best hope of identifying therapeutically effective, non-toxic agents is offered.

The present invention will now be described further by way of example only and with reference to the Figures, which show:

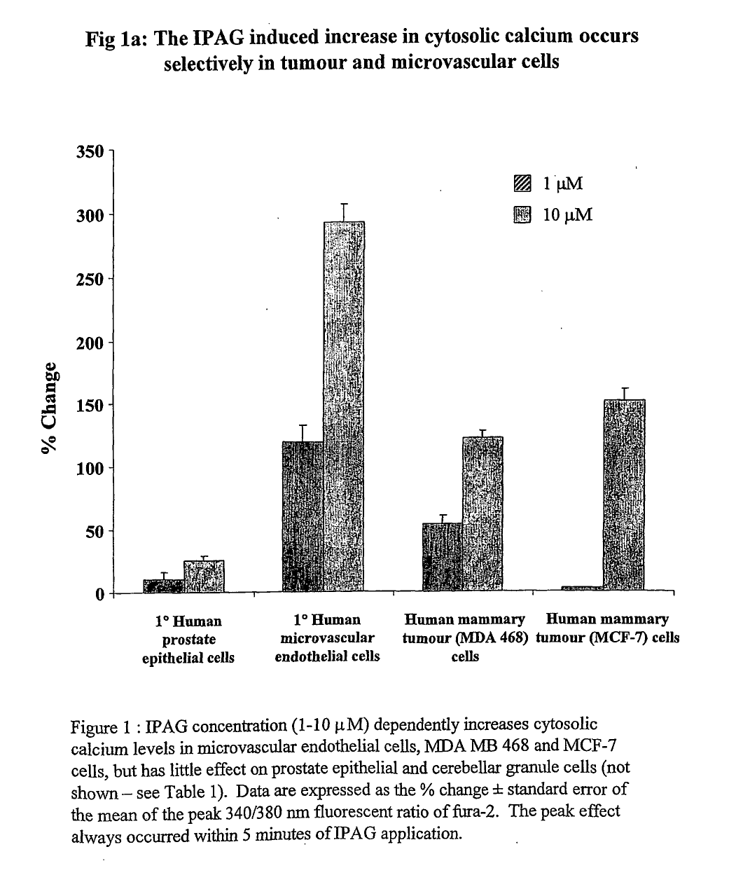

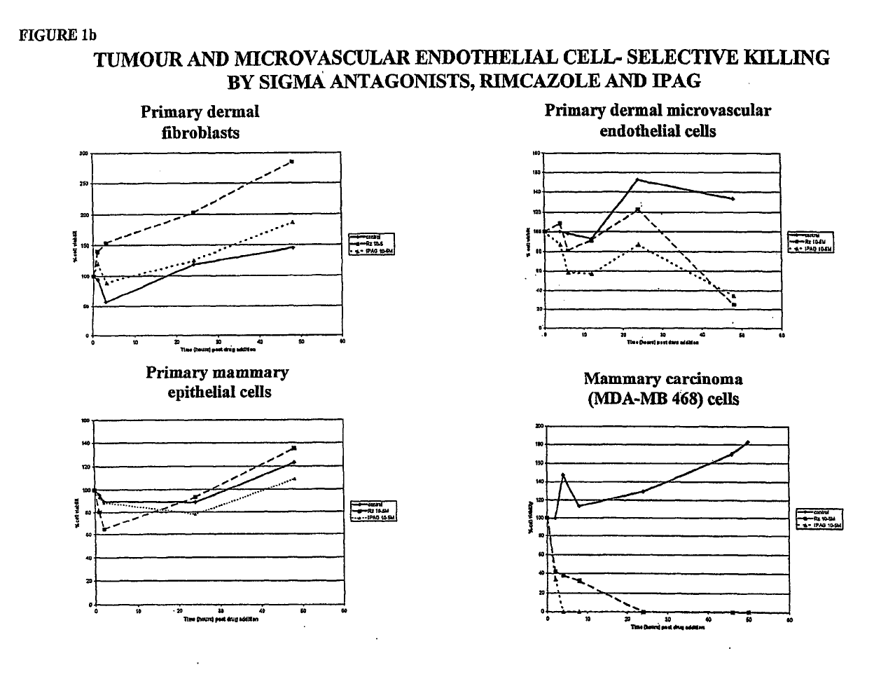

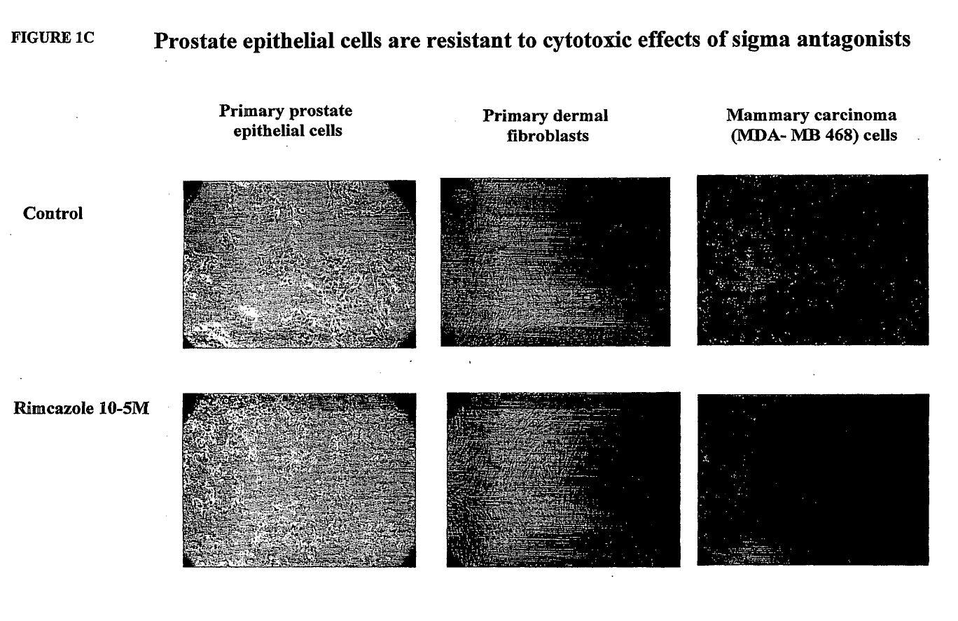

FIG. 1a shows that the IPAG induced increase in cytosolic calcium occurs selectively in tumour and microvascular cells: IPAG concentration (1-10 □M) dependently increases cytosolic calcium levels in microvascular endothelial cells, MDA MB 468 and MCF-7 cells, but has little effect on prostate epithelial and cerebellar granule cells (not shown—see Table 1). Data are expressed as the % change±standard error of the mean of the peak 340/380 nm fluorescent ratio of fura-2. The peak effect always occurred within 5 minutes of IPAG application;

FIG. 1b shows the tumour and microvascular endothelial cell-selective killing by sigma antagonists, rimcazole and IPAG;

FIG. 1c shows that prostate epithelial cells are resistant to cytotoxic effects of sigma antagonists;

FIG. 1d shows that 10 μM of the σ antagonist, IPAG, evokes a rapid increase in cytosolic calcium in MDA MB 468 and MCF-7 cells;

FIG. 1e shows that the IPAG (10 μM) induced increase in cytosolic calcium is sustained in MDA MB 468, but not in MCF-7 cells: IPAG (10 μM) produces an increase in cytosolic calcium in MDA MB 468 and MCF-7 cells. This elevation in calcium is sustained over a period of time in MDA MB 468 cells however, in MCF-7 cells the increase is transient. Data are expressed as the mean±the standard error of the mean of the peak 340/380 nm fluorescent ratio of fura-2;

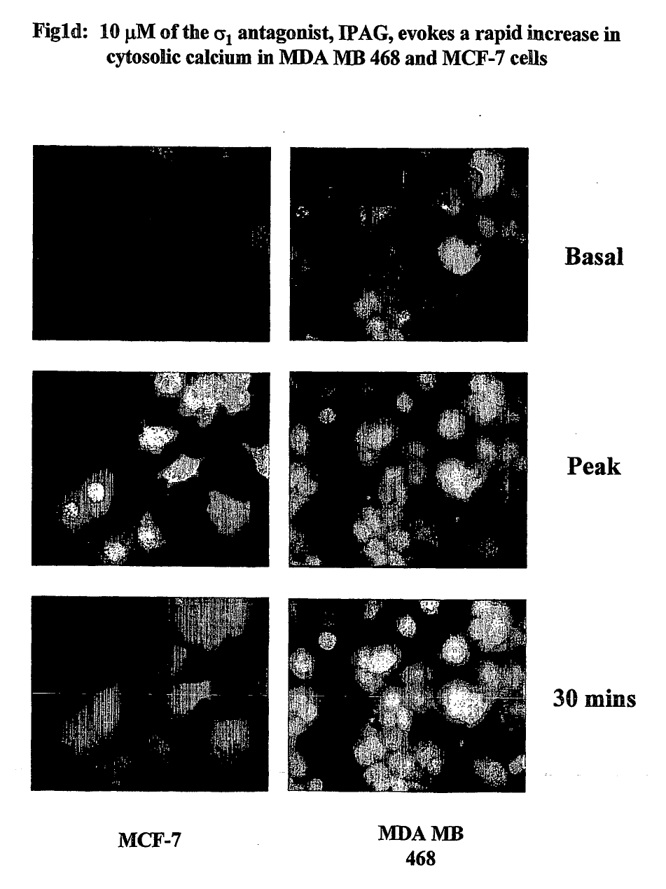

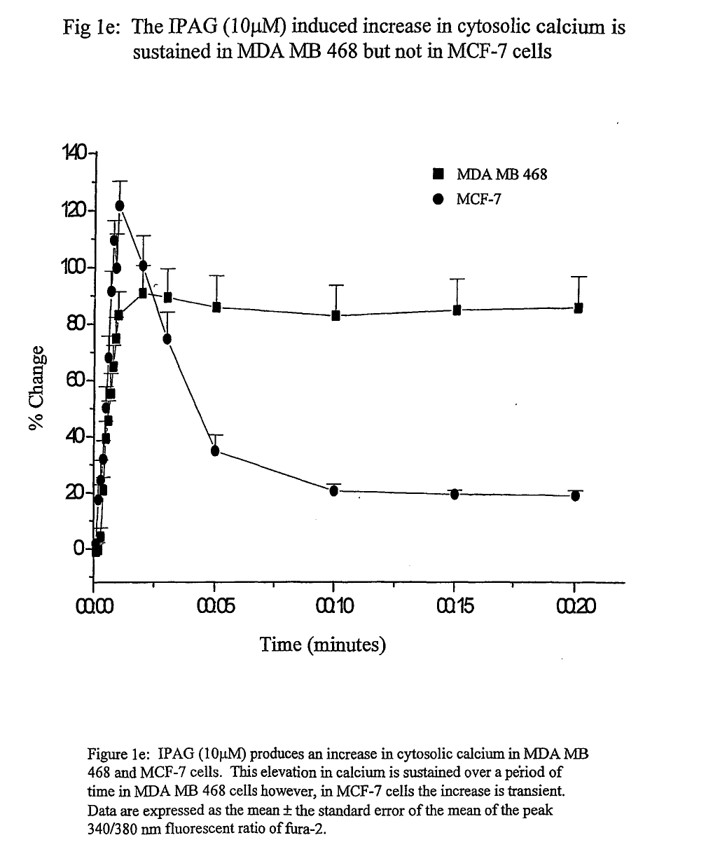

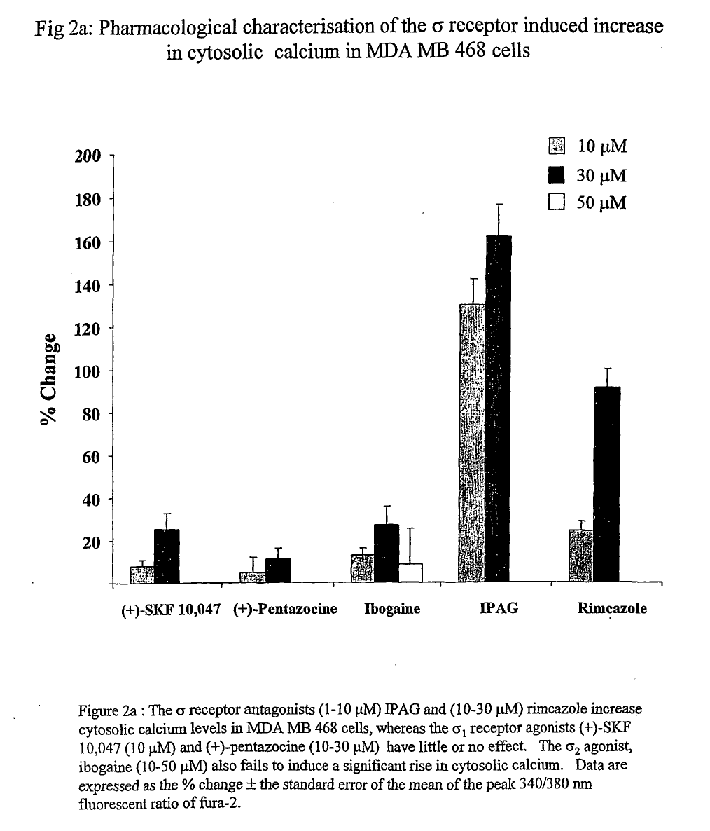

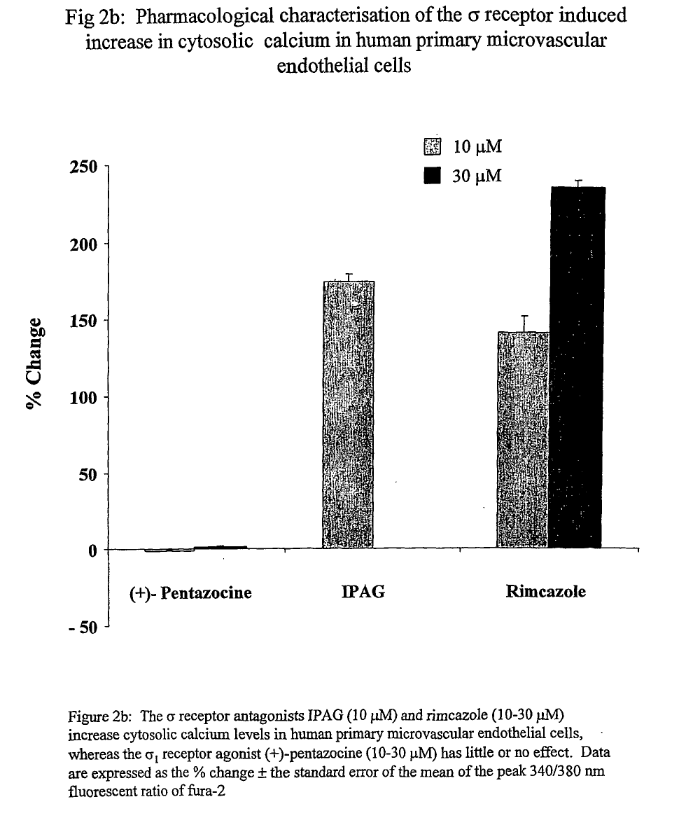

FIG. 2a shows the Pharmacological characterisation of the σ receptor induced increase in cytosolic calcium in MDA MB 468 cells: The σ receptor antagonists (1-10 μM) IPAG and (10-30 μM) rimcazole increase cytosolic calcium levels in MDA MB 468 cells, whereas the σ₁ receptor agonists (+)-SKF 10,047 (10 μM) and (+)-pentazocine (10-30 μM) have little or no effect. The σ₂ agonist, ibogaine (10-50 μM) also fails to induce a significant rise in cytosolic calcium. Data are expressed as the % change±the standard error of the mean of the peak 340/380 nm fluorescent ratio of fura-2;

FIG. 2b shows the Pharmacological characterisation of the σ receptor induced increase in cytosolic calcium in human primary microvascular endothelial cells: The σ receptor antagonists IPAG (10 μM) and rimcazole (10-30 μM) increase cytosolic calcium levels in human primary microvascular endothelial cells, whereas the σ₁ receptor agonist (+)-pentazocine (10-30 μM) has little or no effect. Data are expressed as the % change±the standard error of the mean of the peak 340/380 nm fluorescent ratio of fura-2;

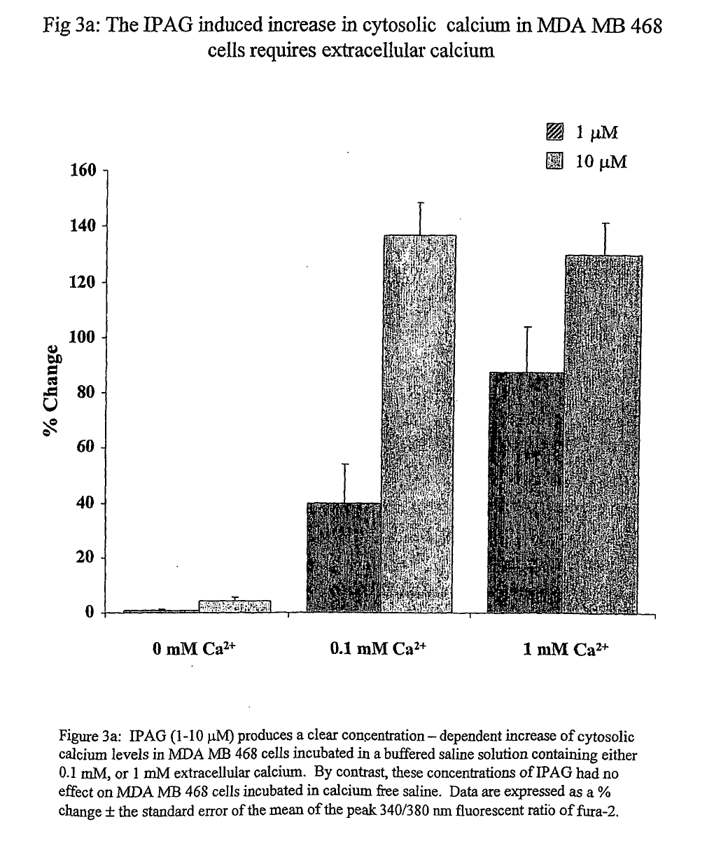

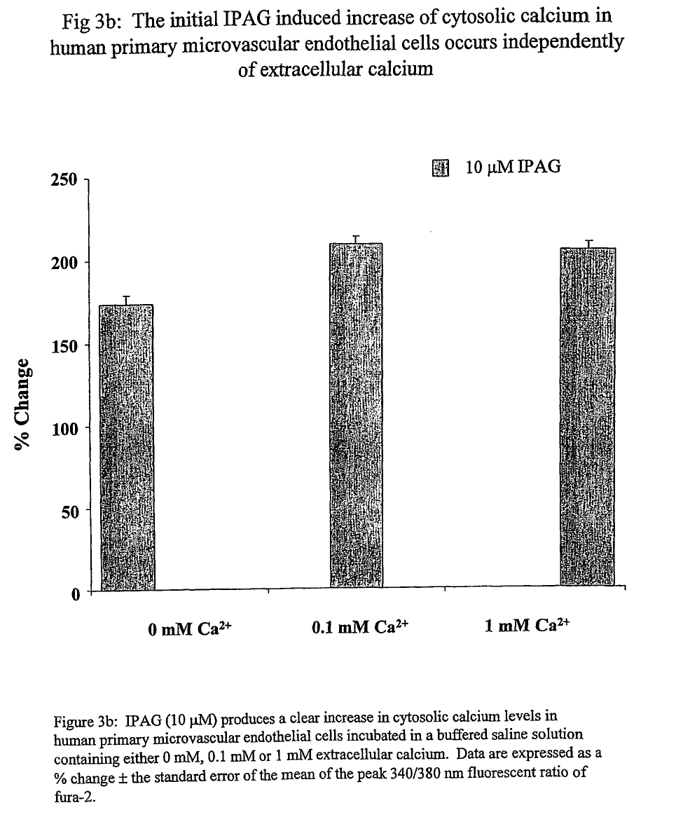

FIG. 3a shows the IPAG induced increase in cytosolic calcium in MDA MB 468 cells requires extracellular calcium: IPAG (1-10 μM produces a clear concentration—dependent increase of cytosolic calcium levels in MDA MB 468 cells incubated in a buffered saline solution containing either 0.1 mM, or 1 mM extracellular calcium. By contrast, these concentrations of IPAG had no effect on MDA MB 468 cells incubated in calcium free saline. Data are expressed as a % change±the standard error of the mean of the peak 340/380 nm fluorescent ratio of fura-2;

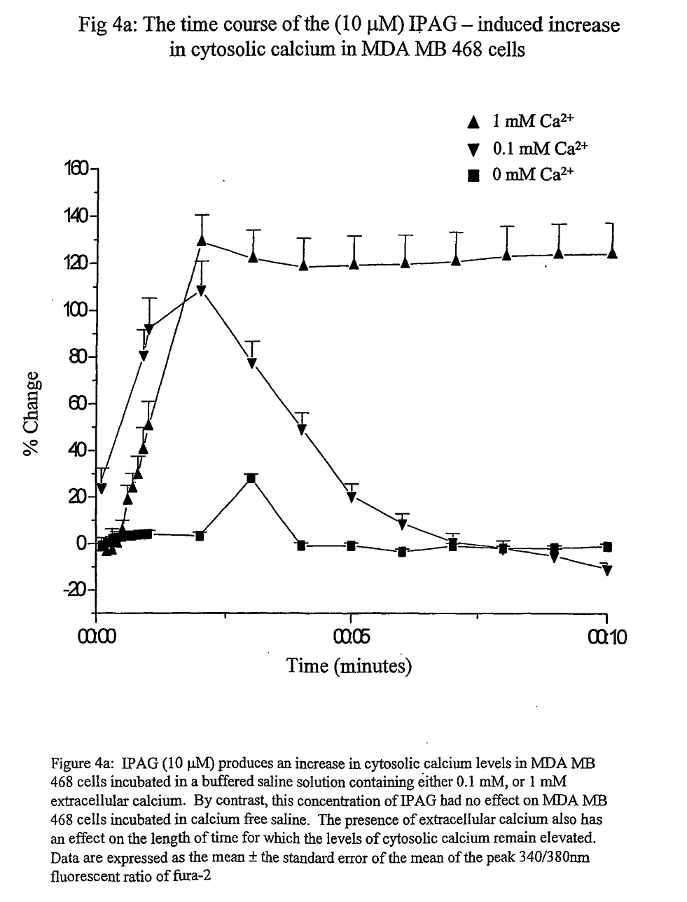

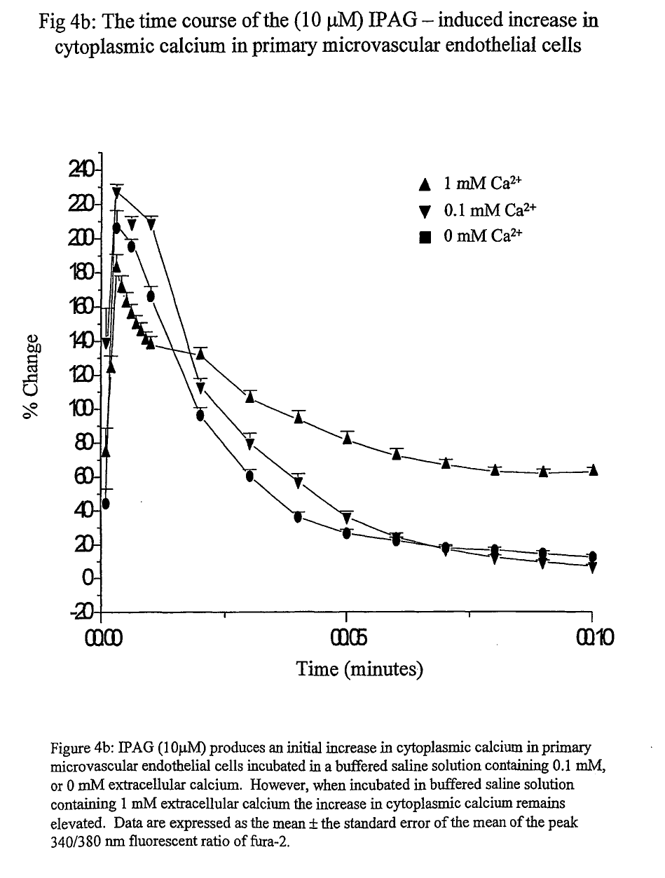

FIG. 3b shows the initial IPAG induced increase of cytosolic calcium in human primary microvascular endothelial cells occurs independently of extracellular calcium IPAG (10 μM) produces a clear increase in cytosolic calcium levels in human primary microvascular endothelial cells incubated in a buffered saline solution containing either 0 mM, 0.1 mM or 1 mM extracellular calcium. Data are expressed as a % change±the standard error of the mean of the peak 340/380 nm fluorescent ratio of fura-2.FIG. 4b: shows the time course of the (10 μM) IPAG—induced increase in cytosolic calcium in primary microvascular endothelial cells: IPAG (10 μM) produces an initial increase in cytoplasmic calcium in primary microvascular endothelial cells incubated in a buffered saline solution containing 0.1 mM, or 0 mM extracellular calcium. However, when incubated in buffered saline solution containing 1 mM extracellular calcium the increase in cytoplasmic calcium remains elevated. Data are expressed as the mean±the standard error of the mean of the peak 340/380 nm fluorescent ratio of fura-2;

FIG. 4a: The time course of the (10 μM) IPAG—induced increase in cytosolic calcium in MDA MB 468 cells: IPAG (10 μM) produces an increase in cytosolic calcium levels in MDA MB 468 cells incubated in a buffered saline solution containing either 0.1 mM, or 1 mM extracellular calcium. By contrast, this concentration of IPAG had no effect on MDA MB 468 cells incubated in calcium free saline. The presence of extracellular calcium also has an effect on the length of time for which the levels of cytosolic calcium remain elevated. Data are expressed as the mean±the standard error of the mean of the peak 340/380 nm fluorescent ratio of fura-2;

FIG. 4b shows The time course of the (10 μM) IPAG—induced increase in cytosolic calcium in primary microvascular endothelial cells: IPAG (10 μM) produces an initial increase in cytosolic calcium in primary microvascular endothelial cells incubated in a buffered saline solution containing 0.1 mM, or 0 mM extracellular calcium. However, when incubated in buffered saline solution containing 1 mM extracellular calcium the increase in cytosolic calcium remains elevated. Data are expressed as the mean±the standard error of the mean of the peak 340/380 nm fluorescent ratio of fura-2;

FIG. 4c shows that MDA-MB 468 cells are inhibited by brief (15-30 mins) exposure to IPAG;

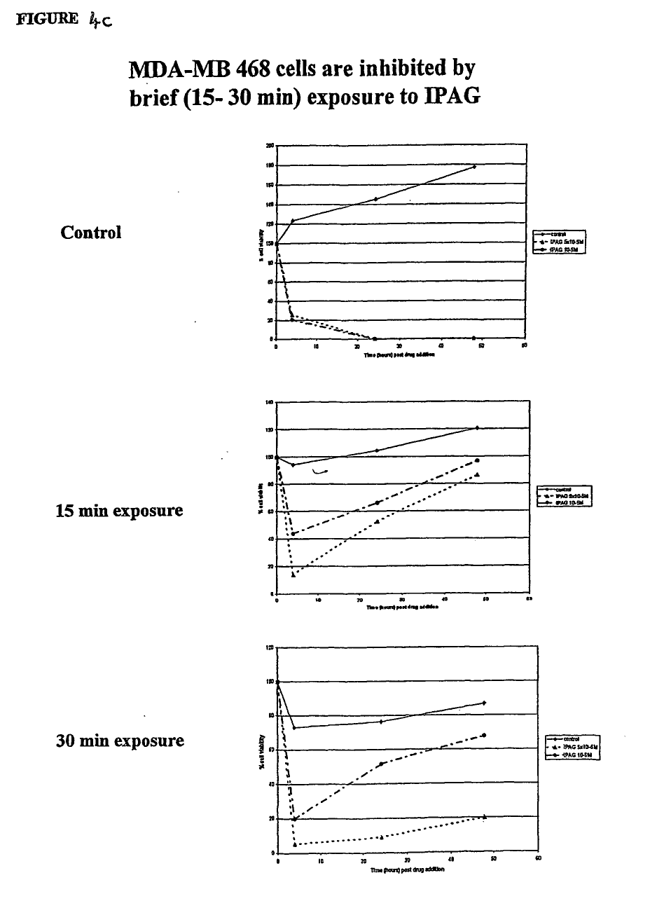

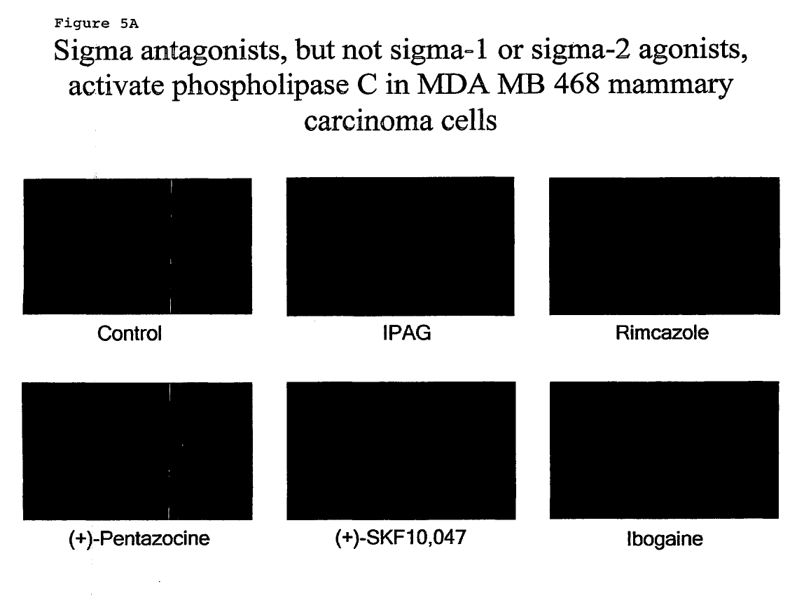

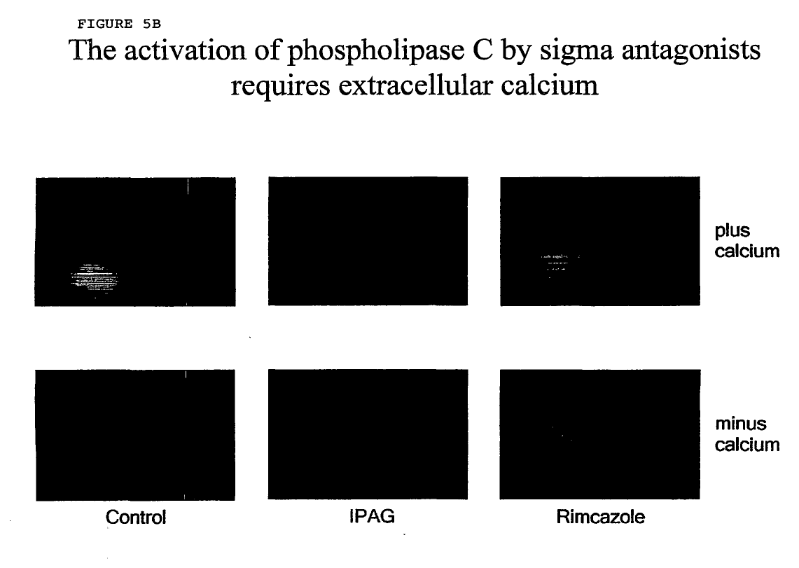

FIG. 5a shows that sigma antagonists, but not sigma-1 or sigma-2 agonists activate phospholipase C in MDA MB 468 mammary carcinoma cells;

FIG. 5b shows the activation of phospholipase C by sigma antagonists requires extracellular calcium;

FIG. 5c shows that the sigma antagonist IPAG inhibits PKB?Akt activity in human mammary carcinoma cells: MDA MB 468 cells maintained in growth medium were treated with either 10 or 100 micromolar IPAG for the times shown and endogenous PKB immunoprecipitated and assayed in vitro. All cells received 1% DMSOvehiclefor the duration of stimulation. Thecontrol experiment shown received 1% DMSO for 30 minutes, butlonger exposure to vehicle does not effect PKB activity. Data pointsshown are the mean PKB activity relative to vehicle treated cells from three independently treated dishes of cells with error barsindicating the standard deviation. As controls, cells were treatedwith the PI 3-kinase inhibitor wortmannin for 30 minutes orstimulated with serum for 5 minutes; and

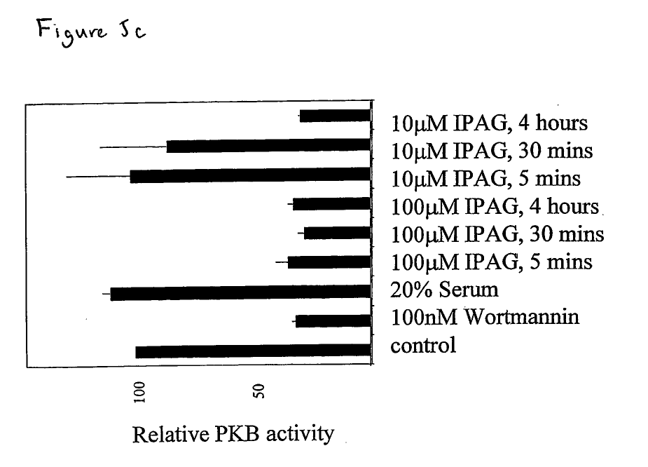

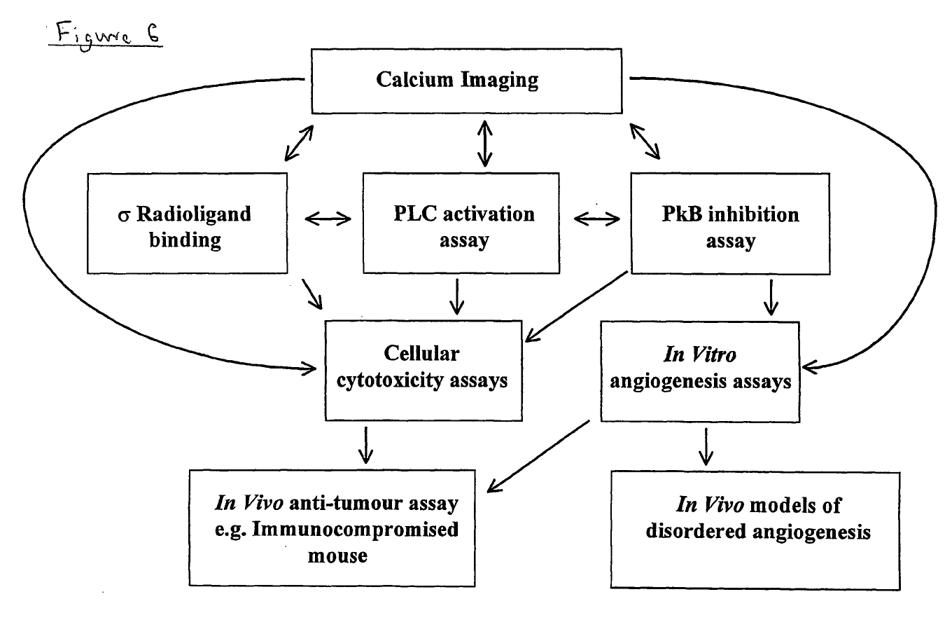

FIG. 6 shows a flow diagram of the assays of the present invention and how these may be combined with other assays/tests.

### MATERIALS AND METHODS SECTION

1. Sigma ligands: Specific sigma ligands—rimcazole, 1-(4-iodophenyl)-3-(2-adamantyl)guanidine IPAG, and (+)-pentazocine (+)-SKF 10,047 and ibogaine—(which have no or minimal cross-reactivity with other known receptors such as mu, delta and kappa opioid, dopamine, serotonin, phencyclidine, and beta-adrenergic receptors) were obtained from Research Biochemicals International (RBI) Inc. (now Sigma/RBI, MO USA)and Tocris Cookson Ltd (MO USA).

Rimcazole is generally regarded as a sigma antagonist; for example Ferris et al (1986 Life Sci Vol 38 pp2329-2337) determined that rimcazole is a specific, competitive antagonist of sigma sites in brain. Rimcazole displays approximately 5-fold selectivity for sigma-1 compared to sigma-2 sites (Abou-Gharbia et al 1993 Annu. Rep. Med. Chem. Vol 28 pp1-10). Thus rimcazole is classed as a sigma-1-preferring antagonist. The compound IPAG has a high affinity for sigma-1 sites (inhibition constant approximately 2.8 nM) and has been described as an antagonist (Whittemore et al 1997 J. Pharm. Exp. Ther. Vol 282 pp326-338).

Whereas antagonistic ligands for the sigma receptor may be less well defined, agonistic ligands which have selectivity for the sigma-1 receptor are generally recognised. Prototypic sigma-1 agonists are (+)pentazocine and (+) SKF 10,047 (e.g. Ceci et al 1988 Eur J Pharmacol Vol 154 pp53-57; Maurice and Privat 1998 Neuroscience Vol 83 pp413-428). Sigma-1 agonists are defined as such on the basis of, for example, stimulation of the brain mesolimbic system (Ceci et al) and potentiation of learning and memory (Maurice and Privat).

Sigma-2 agonists are knoewn in the art (e.g. Vilner and Bowen, 2000). However, the precise definition of sigma ligands as antagonists or agonists must remain provisional until the signal transduction events which mediate the survival-modulating effects of the sigma receptor(s) have been defined.

2. Cell Culture

All primary cells were obtained from Biowhittaker/Clonetics Inc., Walkersville, Md., USA, and grown strictly in accordance with the manufacturer's instructions, using Clonetics specialist media and reagents. (Tissue in all cases was from healthy donors). Independent batches of cells from different donors were studied to ensure reproducibility. Experiments were performed at less than the recommended maximum number of population doublings. Primary cell types included

Human adult dermal microvascular endothelial cells (catalogue number CC-2543; cells had been characterised by tests for cell type markers: positive for acetylated LDL uptake, positive for factor VIII related antigen, negative for alpha smooth muscle actin)

Human mammary epithelial cells (catalogue number CC-2551; stain positive for cytokeratins 14, 18 and 19)

Human prostate epithelial cells (catalogue number CC-2555; stain positive for cytokeratin 8,13.)

Human mammary carcinoma (MDA MB 468) cells were obtained from ATCC, Manassas, Va., USA; catalogue number HTB-132; cells were grown in accordance with the manufacturer's instructions.

Cerebellar granule cells were prepared from 6-7 day post natal rats as previously described (see Courtney, M. J., Lambert, J. J. and Nicholls, D. G. [1990] J. Neuroscience 10: 3873-3879).

3. Cell Viability/Proliferation Assay.

This was carried out using an MTS assay (reagents from Promega Corporation, Madison Wis., USA) which is a modification of the MTT assay (as described in Jacobson et al, 1994 EMBO J Vol 13 pp1899-1910). The assay depends on conversion of the MTS tetrazolium compound to a coloured formazan product in metabolically active cells; it is therefore an assay of viable cell number. A decline in values implies cell killing as long as cell disappearance by apoptosis in untreated cell populations is absent or negligible (as with most healthy primary cell populations).

Cells were seeded in the range 1.5×10⁵-1.8×10⁵ cells per ml of culture medium in 96 well microtitre plates and allowed to attach in a humidifed atmosphere of 5% CO₂ in air at 37 deg C. Drugs were added 18-24 hours later and cell viability/proliferation measured at time intervals up to 48-72 hours post-drug addition when the experiment was terminated. Mean (±SE) values at each time point were obtained from wells in triplicate.

Cell viability was measured as follows: 20 μl of MTS solution (Promega) was added to wells and incubated at 37 deg C. for 3 hours during which time a coloured formazan product is generated in viable cells. (In the MTS assay the formazan product is soluble in tissue culture medium which avoids the solubilisation step required in the MTT assay). Viable cell number was then measured by reading absorbance at 490 nm in a Dynex microtitre plate reader. Cell viability is represented as the ratio of absorbance at time “x” (post drug addition) minus “blank” readings (medium with drug without cells) over absorbance at time zero (prior to drug addition) minus blank readings (medium without drug or cells), expressed as a percentage. 100% reflects viable cell numbers at the start of the experiment; values greater than 100% reflect cell proliferation and values less than 100% reflect cell disappearance (cytotoxicity). These interpretations can be made since values are expressed as a percentage of values at time zero and not relative to control cell populations which will have proliferated in the interim (and therefore increased in cell number).

4. Measurement of Changes in Intracellular Free Calcium Concentration

For calcium imaging experiments cells were plated onto poly-L-lysine coated coverslips (13 mm). Single cell imaging was performed utilizing a MiraCal Imaging facility (Life Science Resources, Cambridge, U.K.) with a Nikon Diaphot-TMD inverted epifluorescence microscope equipped with a ×40 oil immersion objective and Sutter filter wheel. The imager was equipped with a Lambert intensifier 1187 (Life Sciences Resources) providing a ×30 enhancement of the fluorescent signal, thus limiting phototoxicity.

Fura-2 acetoxymethyl ester (fura-2 AM) incubations with the cells were performed at 37° C. in a medium containing (in mM): 120 NaCl, 3.5 KCl, 0.4 KH₂PO₄, 5 NaHCO₃, 1.2 Na₂SO₄, 15 glucose, 1.2 MgCl₂, 1 CaCl₂, and 20 TES [N-tris(hydroxymethyl) methyl-2-aminoethane sulphonic acid] pH adjusted to 7.4 with NaOH. Typically cells were incubated in 3 μM fura-2 AM for 25 min., which permitted sufficient accumulation of free fura-2 in the cell cytoplasm. Cells were then illuminated alternately at 340 and 380 nm with the emitted 505 nm light being detected by a photomultiplier. Free fura emits maximally when excited at 380 nm, whilst the calcium bound fura has a maximal emission when excited at 340 nm. Thus the ratio of emission when alternately excited at 340/380 nm is a measure of the calcium concentration in equilibrium with the chelator. Changes of intracellular calcium were quantified as the percentage increase of the fluorescent ratio (determined at ˜505 nm) obtained by exciting the test cells alternatively at 340 nm (optimal for calcium bound dye) and 380 nm.

Results

Cell-Specific Increases of Cytosolic calcium by IPAG

The sigma receptor antagonist IPAG (1-10 μM) produced an increase of cytosolic calcium in primary microvascular endothelial cells. This effect was relatively rapidly induced, being evident within 5-10 s of extracellular drug application and reaching peak effect within 3 min. The magnitude of the effect was concentration-dependent with 1 μM and 10 μM increasing the calcium ratio by 119±13%, n=47 and 293±14%, n=30, respectively (see FIG. 1a and Table 1). These concentrations of IPAG are cytotoxic in these cells, as is the sigma antagonist rimcazole (FIG. 1b and Table 1).

IPAG produced a similar effect on cytosolic calcium in human mammary tumour (MDA MB 468; hormone insensitive) cells with 1,3 and 10 μM increasing the calcium ratio by 54±7%, n=71; 112±8%, n=32 and 125±7%, n=86 respectively (s FIG. 1a and Table 1). Again these concentrations of IPAG are cytotoxic in MDA 468 cells, as is rimcazole (FIG. 1b, FIG. 1c and Table 1). In hormone sensitive mammary carcinoma (MCF-7) cells 10 μM IPAG induced a similar rise in cytosolic calcium although 1 μM was not effective (FIG. 1a). This is consistent with a lesser degree of cytotoxicity induced by IPAG in MCF-7 cells compared to MDA MB 468 cells.

Although the peak effect on calcium in response to 10 μM IPAG was comparable in MDA MB 468 cells and MCF-7 cells (FIG. 1a) the calcium elevation was sustained for a shorter period in MCF-7 cells. FIG. 1d illustrates that peak effects in response to IPAG occur within 5 minutes in both MCF-7 and MDA MB 468 cells; but calcium levels have returned to baseline in MCF-7 cells by 30 minutes at which time levels in MDA MB 468 cells remain close to peak levels. Thus, the duration of the calcium elevation may also be a factor in determining the degree of cytotoxicity.

What is also evident is that baseline levels of calcium are different in different tumour cell lines (FIG. 1d). MDA MB 468 cells possess high resting calcium levels, which spontaneously oscillate; whereas resting levels of calcium in MCF-7 cells are low. Thus, it is appropriate to represent the change in calcium within cells as the percentage increase relative to baseline levels (as in FIG. 1a) so that differences in resting values can be taken into account.

By contrast, these concentrations (1-10 μM) of IPAG and rimcazole are not cytotoxic in human prostate epithelial cells or in a range of other primary human cells including adult dermal fibroblasts and mammary epithelial cells (FIG. 1b, FIG. 1c and Table 1). In accordance with these findings, 1-10 μM IPAG has little effect on cytosolic calcium in prostate epithelial cells (1 μM=10±5%, n=17; 10 μM=25±5%, n=17 see FIG. 1a and Table 1). The anticipation is that other primary human cells (apart from the special cases such as lens epithelial cells) will also show no or a much lesser calcium response compared to microvascular endothelial cells and tumour cells.

Additionally, IPAG (3 μM) had relatively little effect on the cytosolic calcium levels of cerebellar granule neurones, even though these cells are known to possess sigma binding sites at moderately high density (20±2%, n=13 cf. for MDA468 cells 112±8%, n=32; see Table 1). Thus, the mere presence of sigma binding sites does not determine whether a calcium response to sigma antagonists will occur.

Sigma Receptor Pharmacology of MDA 468 and Microvascular Endothelial Cells

Increases in cytosolic calcium concentrations in tumour cells and microvascular endothelial cells are observed with two compounds—rimcazole and IPAG—which are classed for the purposes of this invention (that is in the context of their ability to modulate cell survival) as sigma-1 antagonists. Existing pharmacological data for these compounds accord with this functional definition but until signal transduction events which mediate survival-modulating effects of the sigma-1 receptor have been defined, existing pharmacological definitions do not necessarily strictly pertain to the functional behaviour in the context of cell survival and proliferation. In contrast, two prototypic sigma-1 agonists (+)-pentazocine and (+)-SKF-10,047 and the sigma-2 agonist, ibogaine, induce little or no rise in cytosolic calcium even in MDA MB 468 cells (FIG. 2a).

In microvascular endothelial cells, there was no detectable rise in cytosolic calcium in response to a prototypic sigma agonist whereas rimcazole and IPAG induced a greater than 100% increase in calcium (FIG. 2b).

For the purposes of this invention, an increase in mean cytosolic calcium of more than 50% above baseline (in the presence of 1 mM extracellular calcium) is deemed to represent a significant elevation of calcium.

Rimcazole and IPAG are defined for the purposes of this invention_as sigma-1 antagonists in light of evidence recently obtained (Spruce et al GB0007842.8, WO 0174359) that rimcazole and IPAG induce cell death and cytostasis in microvascular endothelial cells; death and cytostasis in this cell type is completely prevented (at 10 micromolar concentrations of agents) by equimolar concentrations of the sigma-1 agonist (+) pentazocine. The cloned sigma-1 receptor has also been shown to be anti-apoptotic (Spruce et al GB 0007842.8). Thus, the apoptotic function of rimcazole and IPAG can be deduced to be due to abrogation of sigma-1 mediated survival i.e. rimcazole and IPAG are classed functionally (in this context) as sigma-1 antagonists.

1) Rimcazole is defined by herein (see above) as a sigma-1 receptor antagonist; the prevailing pharmacological view accords with this. This suggests that rimcazole does not behave paradoxically (with reference to its previously described functions in the central nervous system) as an agonist in the context of death induction. Rimcazole produced a concentration-dependent increase of cytosolic calcium levels in MDA 468 cells (30 μM rimcazole=92±8% increase, n=25).(FIG. 2a).

2) There is widespread acceptance that_(+)-pentazocine and (+)-SKF10,047 are sigma-1 receptor agonists and are highly selective for the sigma-1 versus the sigma-2_binding site(eg. Vilner and Bowen, 2000; WO 000599 and WO 0174359). Again, this accords with our functional data that (+) pentazocine stimulates the anti-apoptotic function of the sigma-1 receptor (Spruce et al. GB 0007842.8); thus, (+)pentazocine is behaving as an agonist in the context of both its nervous system and cell survival functions. In contrast to the sigma-1_receptor antagonist IPAG, relatively high concentrations (10-30 μM) of (+)-pentazocine had no significant effect on cytosolic calcium levels of MDA 468 cells (10 μM=5±7%, n=21; 30 μM=−7±6%, n=15)—see FIG. 2.

3) Ibogaine is generally accepted to be a specific sigma-2 agonist (see for example Vilner and Bowen 2000, J. Pharm. Exp. Ther., as cited elsewhere).

Collectively, these observations show that sigma receptor antagonists induce a concentration dependent increase of cytosolic calcium levels in tumour cells and microvascular endothelial cells whereas sigma-1 receptor agonists do not induce such a rise._Sigma-2 agonist—type compounds also fail to induce a significant increase in cytosolic calcium in mammary tumour cells. The previous report by Vilner and Bowen (2000) that ibogaine increases cytosolic calcium in neuroblastoma cells suggests that the calcium response to ibogaine may be tumour type-specific. This invention elucidates elsewhere further points of distinction pertaining to sigma antagonists compared to sigma-2 agonists.

IPAG Requires Extracellular Calcium to Elevate Intracellular Calcium in MDA 468 Cells

The rise in cytosolic calcium induced by sigma-2 -selective ligands in human SK—N—SH neuroblastoma cells occurs in the absence of extracellular calcium (Vilner and Bowen, 2000). Here the present inventors have investigated the influence of extracellular calcium ion concentration on the IPAG induced increase of cytosolic calcium in MDA 468 cells.

In the absence of extracellular calcium, IPAG produced no change in cytosolic calcium levels of MDA 468 cells (1 μM IPAG=1±0.4% increase, n=20; 10 μM=4±2% increase)—see FIG. 3a. By contrast, in the presence of 100 μM extracellular calcium these concentrations of IPAG caused a clear increase of cytosolic calcium (1 μM IPAG=40±14% increase, n=13; 10 μM=237±12% increase, n=16)—see FIG. 3a. Similarly in the presence of lmM extracellular calcium IPAG induced a clear concentration-dependent increase of cytosolic calcium (1 μM IPAG=157±10% increase, n=21; 10 μM=230±12% increase, n=22)—see FIG. 3.

Hence, in respect of a requirement for extracellular calcium to evoke an intracellular calcium rise the effects of the sigma-1 antagonist IPAG on MDA 468 cells differ from those of the sigma-2_receptor ligands in SK—N—SH neuroblastoma cells, as described by Vilner and Bowen (2000).

The Initial Rise in Intracellular Calcium Evoked by IPAG in Microvascular Endothelial Cells Occurs Independently of Extracellular Calcium

Unlike MDA MB 468 cells, the inventors observed that extracellular calcium was not required for the initial rise in intracellular calcium in primary microvascular endothelial cells in response to 10 μM IPAG (FIG. 3b). Initial calcium rises, occurring within 1 minute, in excess of 100% above baseline occurred in the presence of 1 mM and 0.1 mM calcium as well as in nominally calcium free medium (FIGS. 3b and 4b). These data indicate that, at least in microvascular endothelial cells, the initial rise in intracellular calcium is due to release from intracellular stores.

Levels of Extracellular Calcium Affect the Duration of Calcium Elevation in Response to IPAG in Tumour Cells and Microvascular Endothelial Cells

In the presence of 1 mM extracellular calcium, the percentage calcium elevation in response to 10 μM IPAG remains close to peak levels for at least 30 minutes in MDA MB 468 cells. When extracellular calcium is reduced to 0.1 mM, intracellular calcium levels show a progressive decline to baseline within 10 minutes after addition of IPAG (FIG. 4a).

Whereas MDA MB 468 cells retain close to peak cytosolic calcium elevation for at least 30 minutes when extracellular calcium is non-limiting, microvascular endothelial cells resemble MCF-7 mammary tumour cells in showing a progressive decline from peak levels that is evident within approximately 10 minutes, although the percentage elevation is still significant (more than 50% above baseline) at this time. However, in the presence of low (0.1 mM) or nominally absent (0 mM) extracellular calcium, the percentage calcium elevation declines more rapidly so that by 10 minutes, levels are close to or at baseline (FIG. 4b).

These data teach us that the level of extracellular calcium is influential in determining the duration of calcium elevation in both tumour cells and microvascular endothelial cells.

Thus, the assay is ideally to be performed over a time course, in the presence and nominal absence of extracellular calcium, in order that agents of the invention can be more readily distinguished from other agents that elevate intracellular calcium; also, so that variations between tumour cell lines can be accommodated.

A Short Exposure to IPAG, During which Cytosolic Calcium is Elevated, is Sufficient to have an Impact on Cell Survival

In previous studies on IPAG—induced cytotoxicity of MDA 468 cells, the drug was in contact with the cells over the complete experimental period (48 hours). However, as described above, the action of IPAG to induce an increase in cytosolic calcium occurs rapidly (within seconds) and is well maintained over the 30 minute test period of the ion imaging experiment (FIG. 4a). It is the thesis of the inventors that the calcium rise may reflect an early, decision-making event which is not fully transduced into engagement of the death programme until 24-48 hours later. (This is consistent with other stimuli to apoptosis such as inducers of DNA damage which can be fairly immediate in their effect but which do not result in engagement of the death programme until hours or even days later). Indeed, microvascular endothelial cells (FIG. 1b) do not show a significant decline in viability until 24-48 hours after IPAG and rimcazole addition (at 10 micromolar concentrations).

Therefore, to test whether the early period of drug exposure during which the rise in calcium occurs is sufficient to cause a decline in cell viability, the present inventors investigated the effects of relatively brief (15 or 30 min) IPAG incubations on MDA 468 cells, following which cells were washed with buffered saline and then cultured in normal medium without drug. Control cells were washed with buffered saline at the same times but were replenished with medium containing drug which was then left on for the duration of the experiment. Cell viability was measured in the MTS assay as described above, over a time course. FIG. 4c illustrates that MDA MB 468 cells are indeed inhibited by brief (15 and 30 minute) exposures to IPAG. When exposed to IPAG for 15 minutes there is a transient marked decline in MTS values (consistent with a transient decline in mitochondrial function); however, a proportion of cells recover and by 48 hours viable cell number is only 20-30% less than control values. When exposed for 30 minutes, the reduction in viability was more sustained although it was still less than control plates which were exposed to IPAG for 48 hours (upper panel). These data indicate that a short period (15-30 min) of IPAG exposure is sufficient to induce cytotoxicity in mammary tumour cells although the effect was submaximal. This is consistent with the calcium remaining elevated in these cells at 30 minutes. The much lesser effect on cytotoxicity after 15 minutes of exposure to IPAG suggests that the very early rise (within seconds to minutes) in calcium is not so important but rather, the sustained elevation up to and possibly beyond 30 minutes is more important to induce a lasting effect on cell viability.

Hence, the demonstration that even a short exposure to IPAG subsequently induces cytotoxicity in a proportion of MDA MB 468 cells strongly implicates a role for calcium in this effect.

That said, it is likely that calcium, whilst a necessary trigger, may not in itself be sufficient for irreversible engagement of an apoptotic death programme. It is well recognised that the commitment to apoptosis usually occurs downstream from the initiating trigger, such as the point at which cytochrome C is released from mitochondria; however the art is unclear as to when cells are committed to apoptosis and this may vary between different apoptotic stimuli. Thus, it would be expected that a proximal calcium rise, whilst a necessary trigger, does not represent a point of biochemical commitment to death. Subsequent trandsduction events will be required for the cell to reach a “point of no return”. It is therefore possible that downstream modulatory events could rescue cells from death even after a calcium rise occurred.

It is important nonetheless to recognise that a temporal separation between initiating stimulus and point of commitment to death applies to most if not all apoptosis inducers. Thus, notwithstanding the complexities of the biology of apoptosis, the calcium imaging method remains of great value as a screening method that will give a powerful indication of the likelihood that a given agent will induce cytotoxicity (death and/or cytostasis) in diseased and undesirable cells.

As discussed above, papers published in the twelve month period since the filing of this invention indicate that known anti-tumour agents such as TNF induce apoptotic cell death that requires calcium influx mediated by a TRP channel protein. Such agents are not however amongst the agents of the invention. The agents of the invention have a substantially greater therapeutic index (a measure of toxicity for tumour or other diseased or undesirable cells compared to normal cells) in vitro and in vivo (where this has been tested), compared to agents such as TNF. For the purposes of this invention, normal cells are those cell types that have typical properties of survival and proliferation regulation (as described above). The agents of the invention also have the capacity to induce suicide selectively in wholly or partially self-reliant cells such as primary lens epithelial cells and primary microvascular endothelial cells. This functional distinction between TNF and agents of the invention is consistent with the ability of TNF to bind specifically to receptors of the so-called TNF receptor family.

These data nonetheless support the present invention which demonstrates that the cell-selective toxicity of the agents of the invention correlates closely with, and therefore can be predicted by, their ability to cause calcium influx in diseased or undesirable cells.

It was an expectation when the invention was originally filed that at least some cytotoxic agents that have a lesser degree of selectivity compared to the agents of the invention would also induce calcium rises within tumour cells; but that, unlike the agents of the invention, calcium effects would be demonstrated in normal cells. In anticipation of this, the invention in one embodiment required a comparison of the calcium response in normal cells (as defined in this invention) compared to tumour cells, microvascular endothelial cells or persistent inflammatory cells. It was also recognised that normal cells must be tested at low passage (cultured for a short time ex vivo) since sensitivity to sigma antagonists is acquired when primary cells undergo extended passage in tissue culture. This embodiment of the invention is therefore one potential solution to distinguishing agents of the invention from other, more toxic agents that mediate tumour cell death through a calcium-raising mechanism.

It remains desirable however that, if a distinction between agents of the invention and other agents that cause death through raised cytosolic calcium can be made by examination of tumour cells, microvascular endothelial cells or persistent inflammatory cells alone, and without reference to normal cells at low passage, the assay could be simplified. The inventors have therefore devised another embodiment of the invention, based on a biochemical readout of the calcium response to sigma antagonists in tumour cells.

This comprises an assay to detect activation of phospholipase C, with or without an assay to detect inhibition of the PI3 kinase pathway. These assays can be performed in living cells using introduced proteins that consist of pleckstrin homology (PH) domains fused to GFP.

Sigma Antagonists, but not Sigma-1 or Sigma-2 Agonists, Activate Phospholipase C in MDA MB 468 Mammary Carcinoma Cells

The inventors introduced a recombinant plasmid vector encoding a hybrid protein comprised of the pleckstrin homology (PH) domain of phospholipase C (PLC) delta 1 fused green fluorescent protein (GFP) (GFP-PH PLCdelta 1) into MDA MB 468 mammary carcinoma cells. A standard transfection method (Fugene-6—Roche) was used. At approximately 24 hours following the transfection procedure, cells were exposed to the following sigma ligands: the antagonists rimcazole and IPAG; and prototypic sigma-1 agonists (+)-pentazocine and (+)-SKF10,047, as described in this and preceding inventions (BAS #2 and 3). In addition, a prototypic sigma-2 agonist, ibogaine was used. Ibogaine has been described by Vilner and Bowen (2000, J. Pharm. Exp. Ther. Vol 292 pp 900-911). Cells were maintained in DMEM with 10% fetal bovine serum at 37 deg C. in 5% CO2 before and during exposure to sigma ligands which were present at a range of concentrations. Effects on the GFP-PH PLCdelta 1 protein were studied in two ways: by time lapse microscopy (using a Leica inverted fluorescence microscope and a Hammamatsu Orca charge-coupled device camera, linked to an Improvision Open Lab image processing workstation). This enabled the time course of effects to be determined. For provision of higher quality images for display (as in FIG. 5), cells on coverslips were washed with phosphate buffered saline (PBS) and fixed with 3% paraformaldehyde in PBS at time intervals following sigma ligand addition. Control cell populations received drug vehicle alone and were analysed at the same time points.

FIG. 5a illustrates the effect on GFP-PH PLC delta 1 in MDA MB 468 cells after exposure to sigma ligands at a concentration of 100 μM for a period of approximately 10 minutes. Whereas rimcazole and IPAG induced a pronounced relocalisation of the PH domain protein from the membrane to the cytosol within this time period, two prototypic sigma-1 agonists and a sigma-2 agonist, ibogaine, failed to produce this effect. The sigma antagonist—induced cytosolic relocalisation of GFP-PH PLC delta 1 appeared to be maintained until the cells died by apoptosis some hours later. In the presence of sigma-1 and sigma-2 agonists, GFP-PH PLC delta 1 remained associated with the membrane throughout the time course of the experiment. At lower concentrations (10 μM) of sigma antagonists the relocalisation of GFP-PH PLC delta 1 was delayed until approximately 1 hour after sigma ligand addition but was otherwise similar to that induced by higher concentrations of sigma ligands.

The PH domain of PLC delta 1 has a higher affinity for the phosphoinositide IP3 compared to PIP2. IP3 is produced from PIP2 in response to activation of PLC. Since PIP2 is membrane-bound whereas IP3 is cytosolic, the activation of PLC can be visualised in living cells by the relocalisation of GFP-PH PLC delta 1 from membrane to cytosol. A proviso to this interpretation is that, in conditions where PIP2 levels decline relative to IP3, the net effect would be relocalisation of GFP-PH PLC delta 1 from membrane to cytosol. Thus, to exclude this possibility, quantitative phospholipid assays were performed. (In this analysis, cells were labelled with [³H] inositol in inositol-free DMEM (In Vitrogen—Gibco) with dialysed FBS for 48 hours before exposure to sigma ligands. Cells were then lysed and subjected to quantitative HPLC assay). These assays confirmed that IP3 levels had risen in cells exposed to sigma antagonists (IP3 levels had risen to 240% and 166% of the level prior to provision of the test substance in response to IPAG and rimcazole respectively) but had not risen in cells exposed to sigma-1 or sigma-2 agonists. This confirmed activation of PLC by sigma antagonists.

This assay now provides a further means whereby agents of the invention can be distinguished from so-called sigma-2 agonists which have previously been shown to cause release of calcium from intracellular stores and to induce apoptosis (Vilner and Bowen, 2000, as above). However, sigma-2 agonists remain useful as agents with which sigma antagonists can be combined, in order to enhance anti-tumour activity (as described in WO 000599). The pharmacological classification of sigma ligands must however remain provisional until a) the sigma-2 receptor and potentially other sigma receptor subtypes have been cloned; b) signal transduction events that modulate cell survival in response to sigma ligands have been defined; and c) binding pockets—that may mediate discrete functional effects—on each sigma receptor subtype have been defined.

The Activation of Phospholipase C by Sigma Antagonists Requires Extracellular Calcium

The inventors had observed that sigma antagonists fail to induce a rise in cytosolic calcium in MDA MB 468 cells when extracellular calcium was withdrawn. They were therefore interested to know whether the activation of PLC is also calcium dependent. GFP-PH PLC delta 1—expressing MDA MB 468 cells were exposed to sigma antagonists in 1 mM and 0 mM (nominally calcium-free) buffer (as used for the calcium imaging experiments, described above) and monitored by time lapse imaging as above. Cells for image display (FIG. 5b) were fixed at intervals, as above. In the presence of 1 mM calcium-containing buffer, the cytosolic relocalisation of GFP-PH PLC delta 1 in response to sigma antagonists occurred (FIG. 5b, upper middle and right hand panels). However, in nominally calcium-free buffer (0 mM calcium) the GFP-PH PLC delta 1 protein remained membrane-localised in cells exposed to IPAG and rimcazole (FIG. 5b, lower middle and right hand panels).

Thus, the full activation of PLC by sigma antagonists requires the presence of extracellular calcium, from which a requirement for calcium can be inferred. This suggests that sigma antagonists activate the delta 1 isoform of PLC.

IPAG Inhibits PKB/Akt Activity in MDA MB 468 Human Mammary Carcinoma Cells

MDA MB 468 cells lack PTEN, a tumour suppressor gene. This leads to elevated levels of PIP3 and an elevated activation of PKB/Akt which provides a potent survival stimulus to these cells. Given that MDA MB 468 cells are decisively killed when exposed to sigma antagonists, it seemed possible that, in addition to triggering of a calcium/PLC dependent pathway to death, inhibition of PKB might have occurred.

MDA MB 468 cells were exposed to the sigma antagonist IPAG at 100 μM and 10 μM concentrations for periods of time up to 4 hours. At intervals, cells were lysed and then subjected to PKB immunoprecipitation, precisely as has been described previously (Leslie et al., 2001, Biochemical Journal, Vol 357 p427). This method, which is recognised in the art, provides a quantitative measure of PKB activity. FIG. 5c depicts a concentration- and time-dependent decline in PKB activity when cells were exposed to IPAG. 100 μM IPAG profoundly inhibited PKB activity within 5 minutes, an effect that was sustained to at least 30 minutes. 10 μM IPAG induced a later decline in PKB activity (see FIG. 5c)

It has previously demonstrated that sigma receptor ligands induce apoptosis and/or inhibit proliferation in many, possibly all, tumour cell lines, in addition to microvascular endothelial cells, the cell type which gives rise to new blood vessels on which tumour growth and progression are crucially dependent. The inhibition of microvascular endothelial cells was demonstrated on cells grown in isolation from tumour cells; thus, a direct “anti-angiogenic” effect could be claimed (as distinct from an indirect effect due to inhibition of tumour-generated pro-angiogenic factors). Thus, the assay to identify anti-angiogenic agents is performed on pure cultures of microvascular endothelial cells. In contrast, other primary cell types (with typical properties of survival and proliferation regulation) are insensitive or substantially less sensitive to the inhibitory effects of sigma ligands. Hence, such compounds will be novel therapeutic agents in the treatment of cancers as they will induce apoptosis of the tumour directly, but will additionally and directly inhibit the neovascularisation of the tumour, consequently preventing the supply of nutrients to the tumour; yet they will spare most normal cecll types and thus be associated with low toxicity. In vivo exemplification of anti-tumour (WO 00/00599) and anti-angiogenic effects (GB 0007842.8), whilst normal tissues are spared, has been previously obtained. This “two-pronged” selective attack on both the tumour and its vasculature is associated with an amplified anti-tumour effect and a greatly reduced risk of acquired drug resistance since microvascular endothelial cells, unlike the tumour cells themselves, are genetically stable.

Whilst some agents identified in this assay may be equally or comparably effective on tumour and microvascular endothelial ceclls, it is conceivable that a subset of agents may have selective efficacy. The inventors speculate that the sigma-1 receptor may be differentially localised in tumour (transformed) compared to non-transformed cells; thus agents may, through different properties of the chemical backbone be differentially able to access different subcellular pools of the receptor. Agents which are for example selectively effective against microvascular endothelial cells may have enhanced potency and would therefore be of use in non-cancer applications of angiogenesis modulation.

These data clearly demonstrate that the sigma-1 receptor antagonists 1-(4-iodophenyl)-3-(2-adamantyl)guanidine (IPAG) and rimcazole, Induce a rapid, concentration—dependent rise of cytosolic calcium in cells that undergo apoptosis and/or proliferation inhibition in response sigma-1 receptor antagonists but not in cells that are insensitive or substantially less sensitive to sigma antagonists.

These data potentially provide, at least in part, a molecular basis for the pro-apoptotic effects of sigma-1 receptor ligands, but additionally provide a means for a fluorescent ion imaging based high throughput screen for substances that induce cell selective apoptosis and/or inhibition of cell proliferation; also, agents that promote cell survival and/or induction of cell proliferation.

Advantageously, the present inventors have determined that real time calcium imaging is predictive of apoptotic and/or cytostatic outcome in response to sigma ligands, and has a significant advantage over conventional apoptosis and cytotoxicity bioassays as it is rapid and therefore translatable to high throughput screening. Furthermore, high throughput machines for calcium imaging are commercially available eg. VIPR and FLIPR). In conventional apoptosis and cytotoxicity bioassays, the extent of cell death and the timing of engagement of the apoptotic programme are variable since thay are affected for example by cell density and growth state; furthermore, the initiating apoptotic stimulus can precede apoptotic engagement (one measurable outcome in conventional assays) by hours or even days (as described above). This renders it very difficult to use conventional bioassays, certainlyin a high or even medium throughput setting, to predict with any degree of reliability how potent a given sigma ligand may be in the in vivo situation. In contrast, calcium imaging is rapid, reliable and quantitative so will be of great value in providing an objective assessment of sigma ligands which are likely to have anti-tumour (and other medically important) activities in vivo.

Furthermore, calcium imaging is performed on intact, living cells in contrast to classical radioligand binding assays which are performed on isolated cellular membranes. The sigma receptor exists in multiple pools on intracellular as well as cell surface membranes; thus the relative accessibility of a compound to particular subcellular sites may greatly affect its ability to induce apoptosis. This accounts at least in part for why there is sometimes a lack of correlation between binding affinity in radioligand binding assays and apoptotic potency, and emphasises the need for an assay which is quantitative but better able to predict biological outcome than either ligand binding or conventional bioassays.

TABLE 1

| | Concentration of IPAG | | | |
|---|---|---|---|---|
| Cell Type | 1 μM | 3 μM | 10 μM | Cytotoxicity |
| Microvascular | 119 ± 13% | NT | 293 ± 14% | + |
| endothelial | | | | |
| MDA 468 | 54 ± 7% | 112 ± 8% | 125 ± 7% | + |
| MCF-7 | 2.6 ± 0.6% | NT | 135.1 ± 8.8% | + |
| Prostate | 10 ± 5% | NT | 25 ± 5% | − |
| epithelial | | | | |
| Cerebellar | NT | 20 ± 2% | NT | NT |
| granule | | | | |

NT = Not Tested

1) A method for identifying a substance capable of modulating cell survival and/or proliferation, which method comprises:
a) providing a test substance to a cell or cell population that displays abnormal properties of survival and/or proliferation regulation and to a cell or cell population that displays normal properties of cell survival and/or proliferation regulation;
b) observing an effect said test substance has on a level of cytosolic calcium within the abnormal and normal cells; and
c) comprising any effects said test substance has on the level of cytosolic calcium within the abnormal and normal cells, so as to identify agents which affect the level of cytosolic calcium within the abnormal cells whilst minimally or substantially not affecting the level of cytosolic calcium within the normal cells.

2) The method according to claim 1 wherein the level is either absolute or relative to a resting, that is, prior to provision of the test substance, or other reference value.

3) The method according to either of claims 1 or 2 wherein the assay comprises a source of extracellular calcium, is nominally calcium-free, and/or the absence of extracellular calcium.

4) The method according to claim 3, carried out in the presence of physiological (approx 1 mM) calcium for use in identifying agents which cause an increase in cytosolic calcium within abnormal cells, whilst minimally or substantially not affecting the level of cytosolic calcium within normal cells.

5) The method according to claim 4 wherein the increase is at least 50% greater in abnormal cells in comparison to normal cells.

6) The method according to any preceding claim for identifying a novel sigma (antagonistic) ligand.

7) The method according to any preceding claim wherein the abnormal cell or cell population is obtained from a tumour cell or cell line, a lens epithelial, or microvascular endothelial cell of cell line, a cell or cell line in which NFKappaB is dysregulated, a PTEN null cell or cell line or a persistent inflammatory cell or cell line.

8) The method according to any preceding claim wherein the normal cell or cell population is/are obtained from adult fibroblasts cerebellar granule neurones, mammary epithelial cells, or prostate epithelial cells.

9) The method according to any preceding claim wherein any alternation in the level of cytosolic calcium is observed within a few seconds to 10 minutes.

10) The method according to any preceding claim wherein the assay is conducted for 10-60 minutes in order to observe if changes in calcium level over the time course of the assay.

11) The method according to any claim wherein the cytosolic calcium levels are detected by use of fluorescent probes, which shows a spectral response upon binding calcium and which are detected using fluorescence microscopy, flow cytometry fluorescence spectroscopy, a CCD camera, a luminometer, a FLIPR-flumetric imaging based plate reader or a VIPR voltage ion probe reader.

12) The method according to any preceding claim further comprising conducting an assay to determine a degree of PLC activation in said abnormal and/or normal cells.

13) The method according to claim 12 wherein said PLC activation is determined as a consequence of relocalisation of a GFP-PHPLC delta fusion protein and elevation of IP3 and IP4 levels.

14) The method according to either of claims 12 or 13 carried out in the presence of high or low serum levels.

15) The method according to any one of claims 12-14 carried out in the presence, nominal absence and/or absence of extracellular calcium.

16) The method according to any one of claims 12-15 carried out over 48-72 hours.

17) The method according to any preceding claim further comprising the step of detecting the inhibition of PKB activity.

18) The method according to any preceding claim further comprising the step of conducting a sigma radio ligand binding assay.

19) The method according to any preceding claim further comprising the step or step of conducting one or more additional assays/tests as shown in FIG. 6.
